SEQUENCE LISTING (80 sequences)

SEQ ID NO: 1 - DNA, 215 bp, synthetic construct
  misc_feature 1..215: note = synthetic
  source 1..215: mol_type = other DNA
cgtatcagcaggtcccataagctttcgacaattaacgagtgtgtactcgttctatcatctcacagttaaagtcgggagaataggagccgcacgggtcctgataccagcgtgagcggagagaatcttctttctgtctattagtgaagccacagatgtaatagacagaaagaagattctctccgcctacgcccttggcagcaccgtacggttcgatc

SEQ ID NO: 2 - DNA, 121 bp, synthetic construct
  misc_feature 1..121: note = synthetic
  source 1..121: mol_type = other DNA
ctagcgagagaatcttctttctgtctataaaagagagaatcttctttctgtctataaaagagagaatcttctttctgtctataaaagagagaatcttctttctgtctatgtcgaccctgca

SEQ ID NO: 3 - DNA, 286 bp, synthetic construct
  misc_feature 1..286: note = synthetic
  source 1..286: mol_type = other DNA
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggtacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 4 - DNA, 236 bp, synthetic construct
  misc_feature 1..236: note = synthetic
  source 1..236: mol_type = other DNA
tcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 5 - DNA, 202 bp, synthetic construct
  misc_feature 1..202: note = synthetic
  source 1..202: mol_type = other DNA
ggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 6 - DNA, 255 bp, synthetic construct
  misc_feature 1..255: note = synthetic
  source 1..255: mol_type = other DNA
catggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggcc

SEQ ID NO: 7 - DNA, 255 bp, synthetic construct
  misc_feature 1..255: note = synthetic
  source 1..255: mol_type = other DNA
aattcggtacccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaa

SEQ ID NO: 8 - DNA, 1374 bp, synthetic construct
  misc_feature 1..1374: note = synthetic
  source 1..1374: mol_type = other DNA
atggtatcatattgggacacaggagtcctcctgtgcgcccttttgagctgtcttttgctgacgggttcttcttcaggttctgataccgggagaccattcgttgaaatgtatagtgagatacccgaaattatacatatgactgaagggcgcgagctcgtcataccctgtcgggtaacaagtccaaacataacagtgaccctgaagaagttccctttggatactctcattcccgacgggaagcgaataatttgggactcaaggaagggatttatcatatctaatgcaacatataaggaaatagggctcctcacatgcgaagccacagtcaacgggcacctctacaaaactaattacctgacccatcgacagacaaataccatcattgatgtggttctgagtccatctcatggaatagagttgagtgtaggggagaagcttgttctcaattgcacagctagaaccgagttgaacgtgggtatcgattttaactgggaatacccatctagtaagcaccaacacaaaaaacttgtcaatcgagatttgaaaactcaatctggtagcgagatgaaaaagttcctgtcaactcttacaatcgatggcgtgacccggagtgatcaaggattgtatacctgcgccgccagctctggcctgatgactaaaaagaacagcacctttgtacgagtgcatgaaaaggataagactcatacatgccctccttgtcccgctccagagctgctgggaggtcccagtgttttcctcttcccaccaaagcccaaagatactctgatgattagccggacccctgaggtgacttgcgtcgtggtggatgtttcacatgaagatccagaagtgaagttcaattggtacgttgatggtgtggaggtacacaatgccaagactaaacctcgggaggaacagtataacagcacttacagagttgtcagcgtactcacagtgcttcatcaggactggttgaatggtaaggagtataagtgcaaagtgagtaataaggctctgccagcacccatagagaagacaatctcaaaggccaaaggccagccccgagaaccacaagtatacacactgccacctagtagagacgagttgacaaaaaatcaggtcagcctcacctgtctcgtgaaaggcttctaccctagcgacattgccgtagaatgggaaagcaacgggcaaccagaaaacaattataagacaacacctcccgttctcgatagtgacggaagtttcttcctgtatagcaaacttaccgtggataaatcaagatggcagcaaggtaatgtgtttagctgttcagtaatgcacgaagctctgcataaccactacacccaaaaatctttgtctctgtctccagggtga

SEQ ID NO: 9 - DNA, 3325 bp, synthetic construct
  misc_feature 1..3325: note = synthetic
  source 1..3325: mol_type = other DNA
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcaacgcgtattgggatctcgaggcggccgcgccgccaccctagcgagagaatcttctttctgtctataaaagagagaatcttctttctgtctataaaagagagaatcttctttctgtctataaaagagagaatcttctttctgtctatgtcgaccctgcaatggtatcatattgggacacaggagtcctcctgtgcgcccttttgagctgtcttttgctgacgggttcttcttcaggttctgataccgggagaccattcgttgaaatgtatagtgagatacccgaaattatacatatgactgaagggcgcgagctcgtcataccctgtcgggtaacaagtccaaacataacagtgaccctgaagaagttccctttggatactctcattcccgacgggaagcgaataatttgggactcaaggaagggatttatcatatctaatgcaacatataaggaaatagggctcctcacatgcgaagccacagtcaacgggcacctctacaaaactaattacctgacccatcgacagacaaataccatcattgatgtggttctgagtccatctcatggaatagagttgagtgtaggggagaagcttgttctcaattgcacagctagaaccgagttgaacgtgggtatcgattttaactgggaatacccatctagtaagcaccaacacaaaaaacttgtcaatcgagatttgaaaactcaatctggtagcgagatgaaaaagttcctgtcaactcttacaatcgatggcgtgacccggagtgatcaaggattgtatacctgcgccgccagctctggcctgatgactaaaaagaacagcacctttgtacgagtgcatgaaaaggataagactcatacatgccctccttgtcccgctccagagctgctgggaggtcccagtgttttcctcttcccaccaaagcccaaagatactctgatgattagccggacccctgaggtgacttgcgtcgtggtggatgtttcacatgaagatccagaagtgaagttcaattggtacgttgatggtgtggaggtacacaatgccaagactaaacctcgggaggaacagtataacagcacttacagagttgtcagcgtactcacagtgcttcatcaggactggttgaatggtaaggagtataagtgcaaagtgagtaataaggctctgccagcacccatagagaagacaatctcaaaggccaaaggccagccccgagaaccacaagtatacacactgccacctagtagagacgagttgacaaaaaatcaggtcagcctcacctgtctcgtgaaaggcttctaccctagcgacattgccgtagaatgggaaagcaacgggcaaccagaaaacaattataagacaacacctcccgttctcgatagtgacggaagtttcttcctgtatagcaaacttaccgtggataaatcaagatggcagcaaggtaatgtgtttagctgttcagtaatgcacgaagctctgcataaccactacacccaaaaatctttgtctctgtctccagggtgagcgtatcagcaggtcccataagctttcgacaattaacgagtgtgtactcgttctatcatctcacagttaaagtcgggagaataggagccgcacgggtcctgataccagcgtgagcggagagaatcttctttctgtctattagtgaagccacagatgtaatagacagaaagaagattctctccgcctacgcccttggcagcaccgtacggttcgatcagcttattggtacatgataagtctcaggcatcgtacgatgtcgacctgcaggagtcggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 10 - DNA, 27 bp, synthetic construct
  misc_feature 1..27: note = synthetic
  source 1..27: mol_type = other DNA
gagagaatcttctttctgtctataaaa

SEQ ID NO: 11 - DNA, 168 bp, synthetic construct
  misc_feature 1..168: note = synthetic
  source 1..168: mol_type = other DNA
ctcgaggcgatcgcaaacaaacaaagctgtcaccggatgtgctttccggtctgatgagtccgttgtccaataccagcatcgtcttgatgcccttggcagtggatggggacggaggacgaaacagcaaaaagaaaaataaaaattttttttttaattaatcttgggccc

SEQ ID NO: 12 - DNA, 175 bp, synthetic construct
  misc_feature 1..175: note = synthetic
  source 1..175: mol_type = other DNA
ctcgaggcgatcgcaaacaaacaaagctgtcaccggatgtgctttccggtctgatgagtccgttgtccaaaacataccagatttcgatctggagaggtgaagaattcgaccacctggacgaggacggaggacgaaacagcaaaaagaaaaataaaaattaattaatcttgggccc

SEQ ID NO: 13 - DNA, 139 bp, synthetic construct
  misc_feature 1..139: note = synthetic
  source 1..139: mol_type = other DNA
atggccggcatggtcccagcctcctcgctggcgccggctgggcaaccacataccagccgaaaggcccttggcaggtgggcgaatgggacgcacaaatctctctagcttcccagagagaagcgagagaaaagtggctctc

SEQ ID NO: 14 - DNA, 135 bp, synthetic construct
  misc_feature 1..135: note = synthetic
  source 1..135: mol_type = other DNA
atggccggcatggtcccagcctcctcgctggcgccggctgggcaatgctataatcgcgtggatatggcacgcaagtttctaccgggcaccgtaaatgtccgactagtagcgaatgggacgcacaaatctctctag

SEQ ID NO: 15 - DNA, 134 bp, synthetic construct
  misc_feature 1..134: note = synthetic
  source 1..134: mol_type = other DNA
caaacaaacaaaggcgcgtcctggattcgtggtaaaacataccagatttcgatctggagaggtgaagaatacgaccacctgtagtatccagctgatgagtcccaaataggacgaaacgcgctaaacaaacaaac

SEQ ID NO: 16 - DNA, 123 bp, synthetic construct
  misc_feature 1..123: note = synthetic
  source 1..123: mol_type = other DNA
ctgaggtgcaggtacatccagctgacgagtcccaaataggacgaaagggagaggtgaagaatacgaccacctaggctcgaaagagcctaaaacatacctttcctggattccactgctatccac

SEQ ID NO: 17 - DNA, 286 bp, synthetic construct
  misc_feature 1..286: note = synthetic
  source 1..286: mol_type = other DNA
ctagcacgggtccctaaaacataccgtgagcgcgaaagcgcccccattttgagttagtgaagccacagatgtaactcaaaatgggggcgctttcccgcctacggagaggtgaagaatacgaccacctagaagcttattggtacatgataacaccccaaaatcgaagcacttcaaaaacaccccaaaatcgaagcacttcaaaaacaccccaaaatcgaagcacttcaaaaacaccccaaaatcgaagcacttcagtctcaggcatcgtacgatgtcgacctgcagg

SEQ ID NO: 18 - DNA, 1376 bp, synthetic construct
  misc_feature 1..1376: note = synthetic
  source 1..1376: mol_type = other DNA
aatgtccatgttgttctacactctgatcacagcttttctgatcggcatacaggcggaaccacactcagtctgataccgggagaccattcgttgaaatgtatagtgagatacccgaaattatacatatgactgaagggcgcgagctcgtcataccctgtcgggtaacaagtccaaacataacagtgaccctgaagaagttccctttggatactctcattcccgacgggaagcgaataatttgggactcaaggaagggatttatcatatctaatgcaacatataaggaaatagggctcctcacatgcgaagccacagtcaacgggcacctctacaaaactaattacctgacccatcgacagacaaataccatcattgatgtggttctgagtccatctcatggaatagagttgagtgtaggggagaagcttgttctcaattgcacagctagaaccgagttgaacgtgggtatcgattttaactgggaatacccatctagtaagcaccaacacaaaaaacttgtcaatcgagatttgaaaactcaatctggtagcgagatgaaaaagttcctgtcaactcttacaatcgatggcgtgacccggagtgatcaaggattgtatacctgcgccgccagctctggcctgatgactaaaaagaacagcacctttgtacgagtgcatgaaaaggataagactcatacatgccctccttgtcccgctccagagctgctgggaggtcccagtgttttcctcttcccaccaaagcccaaagatactctgatgattagccggacccctgaggtgacttgcgtcgtggtggatgtttcacatgaagatccagaagtgaagttcaattggtacgttgatggtgtggaggtacacaatgccaagactaaacctcgggaggaacagtataacagcacttacagagttgtcagcgtactcacagtgcttcatcaggactggttgaatggtaaggagtataagtgcaaagtgagtaataaggctctgccagcacccatagagaagacaatctcaaaggccaaaggccagccccgagaaccacaagtatacacactgccacctagtagagacgagttgacaaaaaatcaggtcagcctcacctgtctcgtgaaaggcttctaccctagcgacattgccgtagaatgggaaagcaacgggcaaccagaaaacaattataagacaacacctcccgttctcgatagtgacggaagtttcttcctgtatagcaaacttaccgtggataaatcaagatggcagcaaggtaatgtgtttagctgttcagtaatgcacgaagctctgcataaccactacacccaaaaatctttgtctctgtctccagggtgaaaacaaacaaaa

SEQ ID NO: 19 - DNA, 2960 bp, synthetic construct
  misc_feature 1..2960: note = synthetic
  source 1..2960: mol_type = other DNA
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcaacgcgtctcgaggcggccgccgccaccaatggtatcatattgggacacaggagtcctcctgtgcgcccttttgagctgtcttttgctgacgggttcttcttcaggttctgataccgggagaccattcgttgaaatgtatagtgagatacccgaaattatacatatgactgaagggcgcgagctcgtcataccctgtcgggtaacaagtccaaacataacagtgaccctgaagaagttccctttggatactctcattcccgacgggaagcgaataatttgggactcaaggaagggatttatcatatctaatgcaacatataaggaaatagggctcctcacatgcgaagccacagtcaacgggcacctctacaaaactaattacctgacccatcgacagacaaataccatcattgatgtggttctgagtccatctcatggaatagagttgagtgtaggggagaagcttgttctcaattgcacagctagaaccgagttgaacgtgggtatcgattttaactgggaatacccatctagtaagcaccaacacaaaaaacttgtcaatcgagatttgaaaactcaatctggtagcgagatgaaaaagttcctgtcaactcttacaatcgatggcgtgacccggagtgatcaaggattgtatacctgcgccgccagctctggcctgatgactaaaaagaacagcacctttgtacgagtgcatgaaaaggataagactcatacatgccctccttgtcccgctccagagctgctgggaggtcccagtgttttcctcttcccaccaaagcccaaagatactctgatgattagccggacccctgaggtgacttgcgtcgtggtggatgtttcacatgaagatccagaagtgaagttcaattggtacgttgatggtgtggaggtacacaatgccaagactaaacctcgggaggaacagtataacagcacttacagagttgtcagcgtactcacagtgcttcatcaggactggttgaatggtaaggagtataagtgcaaagtgagtaataaggctctgccagcacccatagagaagacaatctcaaaggccaaaggccagccccgagaaccacaagtatacacactgccacctagtagagacgagttgacaaaaaatcaggtcagcctcacctgtctcgtgaaaggcttctaccctagcgacattgccgtagaatgggaaagcaacgggcaaccagaaaacaattataagacaacacctcccgttctcgatagtgacggaagtttcttcctgtatagcaaacttaccgtggataaatcaagatggcagcaaggtaatgtgtttagctgttcagtaatgcacgaagctctgcataaccactacacccaaaaatctttgtctctgtctccagggtgagcggccgcatcggctagcaaagcttcctgcaggagtcggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 20 - DNA, 1374 bp, synthetic construct; identical to SEQ ID NO: 8
  misc_feature 1..1374: note = synthetic
  source 1..1374: mol_type = other DNA

SEQ ID NO: 21 - DNA, 153 bp, synthetic construct
  misc_feature 1..153: note = synthetic
  source 1..153: mol_type = other DNA
gaacgcctcctaacgtctggtcacgaccctgcgtgagcgaccccattgttcaggtttgtattcttttggccactgactgagaatacaaatgaacaatgggggcgcctacgcagcccctcgaaatcacgagggagacgagataagggggcgttt

SEQ ID NO: 22 - DNA, 155 bp, synthetic construct
  misc_feature 1..155: note = synthetic
  source 1..155: mol_type = other DNA
gaacgcctcctaacgtctggtcacgaccctgcgtgagcgaccctgagttccttccctaaatacttttggccactgactgagtatttaggaggaactcaggggggcgcctacgcagcccctcgaaatcacgagggagacgagataagggggcgttt

SEQ ID NO: 23 - DNA, 155 bp, synthetic construct
  misc_feature 1..155: note = synthetic
  source 1..155: mol_type = other DNA
gaacgcctcctaacgtctggtcacgaccctgcgtgagcgaccaagagttaacgtgtcgatctcttttggccactgactgagagatcgacgttaactcttgggggcgcctacgcagcccctcgaaatcacgagggagacgagataagggggcgttt

SEQ ID NO: 24 - DNA, 155 bp, synthetic construct
  misc_feature 1..155: note = synthetic
  source 1..155: mol_type = other DNA
gaacgcctcctaacgtctggtcacgaccctgcgtgagcgacccttccctaaatagtatagatcttttggccactgactgagatctatactttagggaaggggggcgcctacgcagcccctcgaaatcacgagggagacgagataagggggcgttt

SEQ ID NO: 25 - DNA, 155 bp, synthetic construct
  misc_feature 1..155: note = synthetic
  source 1..155: mol_type = other DNA
gaacgcctcctaacgtctggtcacgaccctgcgtgagcgaccgtgtcgatcttggctcaactcttttggccactgactgagcgtgtcgattggctcaactggggcgcctacgcagcccctcgaaatcacgagggagacgagataagggggcgttt

SEQ ID NO: 26 - DNA, 127 bp, synthetic construct
  misc_feature 1..127: note = synthetic
  source 1..127: mol_type = other DNA
gaacgcctccttgataccagctgcgtgagcgaccccattgttcaggtttgtattcttttggccactgactgagaatacaaatgaacaatgggggcgcctacgcgcccttggcagcaagggggcgttt

SEQ ID NO: 27 - DNA, 129 bp, synthetic construct
  misc_feature 1..129: note = synthetic
  source 1..129: mol_type = other DNA
gaacgcctccttgataccagctgcgtgagcgaccctgagttccttccctaaatacttttggccactgactgagtatttaggaggaactcaggggggcgcctacgcgcccttggcagcaagggggcgttt

SEQ ID NO: 28 - DNA, 129 bp, synthetic construct
  misc_feature 1..129: note = synthetic
  source 1..129: mol_type = other DNA
gaacgcctccttgataccagctgcgtgagcgaccaagagttaacgtgtcgatctcttttggccactgactgagagatcgacgttaactcttgggggcgcctacgcgcccttggcagcaagggggcgttt

SEQ ID NO: 29 - DNA, 129 bp, synthetic construct
  misc_feature 1..129: note = synthetic
  source 1..129: mol_type = other DNA
gaacgcctccttgataccagctgcgtgagcgacccttccctaaatagtatagatcttttggccactgactgagatctatactttagggaaggggggcgcctacgcgcccttggcagcaagggggcgttt

SEQ ID NO: 30 - DNA, 129 bp, synthetic construct
  misc_feature 1..129: note = synthetic
  source 1..129: mol_type = other DNA
gaacgcctccttgataccagctgcgtgagcgaccgtgtcgatcttggctcaactcttttggccactgactgagcgtgtcgattggctcaactggggcgcctacgcgcccttggcagcaagggggcgttt

SEQ ID NO: 31 - DNA, 151 bp, synthetic construct
  misc_feature 1..151: note = synthetic
  source 1..151: mol_type = other DNA
gaacgcctcctagaggtgaagaatacgaccaccttgcgtgagcgaccccattgttcaggtttgtattcttttggccactgactgagaatacaaatgaacaatgggggcgcctacgcaggctcgaaagagcctaaaacataagggggcgttt

SEQ ID NO: 32 - DNA, 153 bp, synthetic construct
  misc_feature 1..153: note = synthetic
  source 1..153: mol_type = other DNA
gaacgcctcctagaggtgaagaatacgaccaccttgcgtgagcgaccctgagttccttccctaaatacttttggccactgactgagtatttaggaggaactcaggggggcgcctacgcaggctcgaaagagcctaaaacataagggggcgttt

SEQ ID NO: 33 - DNA, 153 bp, synthetic construct
  misc_feature 1..153: note = synthetic
  source 1..153: mol_type = other DNA
gaacgcctcctagaggtgaagaatacgaccaccttgcgtgagcgaccaagagttaacgtgtcgatctcttttggccactgactgagagatcgacgttaactcttgggggcgcctacgcaggctcgaaagagcctaaaacataagggggcgttt

SEQ ID NO: 34 - DNA, 153 bp, synthetic construct
  misc_feature 1..153: note = synthetic
  source 1..153: mol_type = other DNA
gaacgcctcctagaggtgaagaatacgaccaccttgcgtgagcgacccttccctaaatagtatagatcttttggccactgactgagatctatactttagggaaggggggcgcctacgcaggctcgaaagagcctaaaacataagggggcgttt

SEQ ID NO: 35 - DNA, 153 bp, synthetic construct
  misc_feature 1..153: note = synthetic
  source 1..153: mol_type = other DNA
gaacgcctcctagaggtgaagaatacgaccaccttgcgtgagcgaccgtgtcgatcttggctcaactcttttggccactgactgagcgtgtcgattggctcaactggggcgcctacgcaggctcgaaagagcctaaaacataagggggcgttt

SEQ ID NO: 36 - DNA, 2077 bp, synthetic construct
  misc_feature 1..2077: note = synthetic
  source 1..2077: mol_type = other DNA
aatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaaaaacaaacaaaa

SEQ ID NO: 37 - DNA, 155 bp, synthetic construct
  misc_feature 1..155: note = synthetic
  source 1..155: mol_type = other DNA
gaacgcctcctaacgtctggtcacgaccctgcgtgagcgaccagacgaagagtgtcctagatcttttggccactgactgagatctaggactcttcgtctgggggcgcctacgcagcccctcgaaatcacgagggagacgagataagggggcgttt

SEQ ID NO: 38 - DNA, 155 bp, synthetic construct
  misc_feature 1..155: note = synthetic
  source 1..155: mol_type = other DNA
gaacgcctcctaacgtctggtcacgaccctgcgtgagcgaccgctttccgactcgtattgatcttttggccactgactgagatcaatacgtcggaaagcgggggcgcctacgcagcccctcgaaatcacgagggagacgagataagggggcgttt

SEQ ID NO: 39 - DNA, 155 bp, synthetic construct
  misc_feature 1..155: note = synthetic
  source 1..155: mol_type = other DNA
gaacgcctcctaacgtctggtcacgaccctgcgtgagcgaccaccttctttaccgtttgttacttttggccactgactgagtaacaaactaaagaaggtgggggcgcctacgcagcccctcgaaatcacgagggagacgagataagggggcgttt

SEQ ID NO: 40 - DNA, 155 bp, synthetic construct
  misc_feature 1..155: note = synthetic
  source 1..155: mol_type = other DNA
gaacgcctcctaacgtctggtcacgaccctgcgtgagcgacccgtttgttaagacgtcatgacttttggccactgactgagtcatgacgttaacaaacggggggcgcctacgcagcccctcgaaatcacgagggagacgagataagggggcgttt

SEQ ID NO: 41 - DNA, 155 bp, synthetic construct
  misc_feature 1..155: note = synthetic
  source 1..155: mol_type = other DNA
gaacgcctcctaacgtctggtcacgaccctgcgtgagcgacgcaaatatctagctgtacctacttttggccactgactgagtaggtacatagatatttgcggggcgcctacgcagcccctcgaaatcacgagggagacgagataagggggcgttt

SEQ ID NO: 42 - DNA, 129 bp, synthetic construct
  misc_feature 1..129: note = synthetic
  source 1..129: mol_type = other DNA
gaacgcctccttgataccagctgcgtgagcgaccagacgaagagtgtcctagatcttttggccactgactgagatctaggactcttcgtctgggggcgcctacgcgcccttggcagcaagggggcgttt

SEQ ID NO: 43 - DNA, 129 bp, synthetic construct
  misc_feature 1..129: note = synthetic
  source 1..129: mol_type = other DNA
gaacgcctccttgataccagctgcgtgagcgaccgctttccgactcgtattgatcttttggccactgactgagatcaatacgtcggaaagcgggggcgcctacgcgcccttggcagcaagggggcgttt

SEQ ID NO: 44 - DNA, 129 bp, synthetic construct
  misc_feature 1..129: note = synthetic
  source 1..129: mol_type = other DNA
gaacgcctccttgataccagctgcgtgagcgaccaccttctttaccgtttgttacttttggccactgactgagtaacaaactaaagaaggtgggggcgcctacgcgcccttggcagcaagggggcgttt

SEQ ID NO: 45 - DNA, 129 bp, synthetic construct
  misc_feature 1..129: note = synthetic
  source 1..129: mol_type = other DNA
gaacgcctccttgataccagctgcgtgagcgacccgtttgttaagacgtcatgacttttggccactgactgagtcatgacgttaacaaacggggggcgcctacgcgcccttggcagcaagggggcgttt

SEQ ID NO: 46 - DNA, 129 bp, synthetic construct
  misc_feature 1..129: note = synthetic
  source 1..129: mol_type = other DNA
gaacgcctccttgataccagctgcgtgagcgacgcaaatatctagctgtacctacttttggccactgactgagtaggtacatagatatttgcggggcgcctacgcgcccttggcagcaagggggcgttt

SEQ ID NO: 47 - DNA, 153 bp, synthetic construct
  misc_feature 1..153: note = synthetic
  source 1..153: mol_type = other DNA
gaacgcctcctagaggtgaagaatacgaccaccttgcgtgagcgaccagacgaagagtgtcctagatcttttggccactgactgagatctaggactcttcgtctgggggcgcctacgcaggctcgaaagagcctaaaacataagggggcgttt

SEQ ID NO: 48 - DNA, 153 bp, synthetic construct
  misc_feature 1..153: note = synthetic
  source 1..153: mol_type = other DNA
gaacgcctcctagaggtgaagaatacgaccaccttgcgtgagcgaccgctttccgactcgtattgatcttttggccactgactgagatcaatacgtcggaaagcgggggcgcctacgcaggctcgaaagagcctaaaacataagggggcgttt

SEQ ID NO: 49 - DNA, 153 bp, synthetic construct
  misc_feature 1..153: note = synthetic
  source 1..153: mol_type = other DNA
gaacgcctcctagaggtgaagaatacgaccaccttgcgtgagcgaccaccttctttaccgtttgttacttttggccactgactgagtaacaaactaaagaaggtgggggcgcctacgcaggctcgaaagagcctaaaacataagggggcgttt

SEQ ID NO: 50 - DNA, 153 bp, synthetic construct
  misc_feature 1..153: note = synthetic
  source 1..153: mol_type = other DNA
gaacgcctcctagaggtgaagaatacgaccaccttgcgtgagcgacccgtttgttaagacgtcatgacttttggccactgactgagtcatgacgttaacaaacggggggcgcctacgcaggctcgaaagagcctaaaacataagggggcgttt

SEQ ID NO: 51 - DNA, 142 bp, synthetic construct
  misc_feature 1..142: note = synthetic
  source 1..142: mol_type = other DNA
gaacgcctcctagaggtgaagaatacgaccaccttgcgtgagcgacgcaaatatctagctgtacctacttttggccactgactgagtaggtacatagatatttgcggggcgcctacgcaggctcgaaagagcctaaaacata

SEQ ID NO: 52 - DNA, 1815 bp, synthetic construct
  misc_feature 1..1815: note = synthetic
  source 1..1815: mol_type = other DNA
atgcttgcccgggccctgcttctgtgtgcggtattggcactgagtcacacagccaacccgtgctgctcacacccatgtcagaatcggggagtttgcatgtcagtagggtttgaccaatataagtgcgactgtacccggacaggcttttatggagaaaattgttctacacccgaatttttgacgaggattaagctctttctgaagcctacaccaaatacagtccattatatcctcacgcacttcaagggtttttggaatgtcgtaaacaacattccgtttctgagaaatgcgataatgtcttacgtccttacatcccggagccaccttattgactcacccccaacatacaacgccgactacggatacaaaagttgggaagctttctccaacctgtcttactacacgcgagcattgcctcctgtacccgacgattgcccgacgccgttgggggtgaaaggaaaaaagcagctcccggactcaaacgaaatagtagagaaattgttgcttcgccgaaaatttataccagacccgcagggtagcaatatgatgtttgcgttcttcgctcagcacttcactcaccaattctttaagacagaccacaagagaggtcccgcattcacgaatggccttggacacggcgtcgacttgaaccacatatatggggagacattggccagacaaaggaaacttcgattgtttaaggatgggaaaatgaaataccaaattattgatggcgagatgtatcctccaacagtgaaggatacgcaagcggagatgatttatccaccgcaggtaccagagcatttgcggttcgcggtggggcaggaagtatttggacttgttcccggtctgatgatgtacgccacaatatggctccgggagcataatcgcgtgtgtgacgtacttaagcaagagcacccggaatggggcgacgagcaactttttcaaacgagtcgactgattctcattggggaaacgattaaaatagtgatcgaagattatgtgcaacatctttctggttaccactttaagttgaagttcgatcctgagctgcttttcaataaacaattccagtatcaaaacagaatcgccgcggagttcaacacgctgtatcattggcatcccctgttgcccgatacttttcaaatccatgaccagaaatacaattaccagcaatttatatataataactctatcctcctggagcatggcattacacaattcgttgaaagtttcacaaggcagatcgcaggaagagttgcgggtggccggaatgtgccgcctgccgtgcaaaaggtatcacaggctagcatcgaccaatcaaggcaaatgaagtaccagtcttttaatgagtacaggaaaaggttcatgctgaagccttacgagagctttgaagaactgacgggcgaaaaggaaatgtccgcggaactggaagcgttgtacggagatattgatgcggtagagctctaccccgcgcttctggtcgaaaagccccggccagatgccattttcggggaaaccatggttgaagtgggcgccccattcagtttgaaaggtctgatgggtaatgtaatttgcagtccggcgtactggaagccttccacttttggtggggaagtggggtttcaaattatcaatacggcctcaattcagtctctgatttgtaacaatgttaagggatgtccctttacatcttttagtgtaccggaccccgagcttattaagaccgtgaccataaatgcttcaagctctagaagtggtcttgatgatatcaaccccacagttttgcttaaggaaaggagcacggagctctaa

SEQ ID NO: 53 - DNA, 894 bp, synthetic construct
  misc_feature 1..894: note = synthetic
  source 1..894: mol_type = other DNA
atgagcatgaacaactcaaaacagttggtctccccagcagcggcccttttgtccaacacaacgtgtcagactgagaatagactcagtgtattttttagcgtgatttttatgaccgtcggtatcctgtctaactctcttgccatagcaatattgatgaaagcctatcagcgattccggcaaaagtctaaggcgtcatttttgttgttggcaagcgggcttgtcatcacggactttttcgggcacctcattaacggagccatagccgtgttcgtttatgcttcagacaaagagtggatacgattcgatcagagcaatgtgctttgcagtatctttggcatatgtatggtgtttagtggattgtgtccactcttgcttgggagcgtgatggctattgaaagatgcatcggggtgactaaacctatctttcacagtaccaaaattacatctaagcacgtcaagatgatgttgtccggggtgtgtctttttgctgtctttattgcgttgttgccaatactgggccacagggactacaaaattcaggcctcacgcacttggtgcttctataacactgaggatattaaagactgggaggataggttttatcttcttcttttctcattcctcggtctgctcgcactgggggtatcccttttgtgtaacgctatcacaggtatcaccttgttgcgcgttaaatttaagagccaacagcatcgccaggggaggtcacatcacctcgaaatggtaatacagttgctcgcgatcatgtgtgttagttgtatctgctggtcacccttcttgggatatagaataattctgaatggaaaagaaaagtacaaagtctatgaagaacagagcgattttctgcatcgattgcaatggcccactttggaataa

SEQ ID NO: 54 - DNA, 2771 bp, synthetic construct
  misc_feature 1..2771: note = synthetic
  source 1..2771: mol_type = other DNA
aatgcttgcccgggccctgcttctgtgtgcggtattggcactgagtcacacagccaacccgtgctgctcacacccatgtcagaatcggggagtttgcatgtcagtagggtttgaccaatataagtgcgactgtacccggacaggcttttatggagaaaattgttctacacccgaatttttgacgaggattaagctctttctgaagcctacaccaaatacagtccattatatcctcacgcacttcaagggtttttggaatgtcgtaaacaacattccgtttctgagaaatgcgataatgtcttacgtccttacatcccggagccaccttattgactcacccccaacatacaacgccgactacggatacaaaagttgggaagctttctccaacctgtcttactacacgcgagcattgcctcctgtacccgacgattgcccgacgccgttgggggtgaaaggaaaaaagcagctcccggactcaaacgaaatagtagagaaattgttgcttcgccgaaaatttataccagacccgcagggtagcaatatgatgtttgcgttcttcgctcagcacttcactcaccaattctttaagacagaccacaagagaggtcccgcattcacgaatggccttggacacggcgtcgacttgaaccacatatatggggagacattggccagacaaaggaaacttcgattgtttaaggatgggaaaatgaaataccaaattattgatggcgagatgtatcctccaacagtgaaggatacgcaagcggagatgatttatccaccgcaggtaccagagcatttgcggttcgcggtggggcaggaagtatttggacttgttcccggtctgatgatgtacgccacaatatggctccgggagcataatcgcgtgtgtgacgtacttaagcaagagcacccggaatggggcgacgagcaactttttcaaacgagtcgactgattctcattggggaaacgattaaaatagtgatcgaagattatgtgcaacatctttctggttaccactttaagttgaagttcgatcctgagctgcttttcaataaacaattccagtatcaaaacagaatcgccgcggagttcaacacgctgtatcattggcatcccctgttgcccgatacttttcaaatccatgaccagaaatacaattaccagcaatttatatataataactctatcctcctggagcatggcattacacaattcgttgaaagtttcacaaggcagatcgcaggaagagttgcgggtggccggaatgtgccgcctgccgtgcaaaaggtatcacaggctagcatcgaccaatcaaggcaaatgaagtaccagtcttttaatgagtacaggaaaaggttcatgctgaagccttacgagagctttgaagaactgacgggcgaaaaggaaatgtccgcggaactggaagcgttgtacggagatattgatgcggtagagctctaccccgcgcttctggtcgaaaagccccggccagatgccattttcggggaaaccatggttgaagtgggcgccccattcagtttgaaaggtctgatgggtaatgtaatttgcagtccggcgtactggaagccttccacttttggtggggaagtggggtttcaaattatcaatacggcctcaattcagtctctgatttgtaacaatgttaagggatgtccctttacatcttttagtgtaccggaccccgagcttattaagaccgtgaccataaatgcttcaagctctagaagtggtcttgatgatatcaaccccacagttttgcttaaggaaaggagcacggagctcggaagcggagctactaacttcagcctgctgaagcaggctggagacgtcgaggagaaccctggacctagcatgaacaactcaaaacagttggtctccccagcagcggcccttttgtccaacacaacgtgtcagactgagaatagactcagtgtattttttagcgtgatttttatgaccgtcggtatcctgtctaactctcttgccatagcaatattgatgaaagcctatcagcgattccggcaaaagtctaaggcgtcatttttgttgttggcaagcgggcttgtcatcacggactttttcgggcacctcattaacggagccatagccgtgttcgtttatgcttcagacaaagagtggatacgattcgatcagagcaatgtgctttgcagtatctttggcatatgtatggtgtttagtggattgtgtccactcttgcttgggagcgtgatggctattgaaagatgcatcggggtgactaaacctatctttcacagtaccaaaattacatctaagcacgtcaagatgatgttgtccggggtgtgtctttttgctgtctttattgcgttgttgccaatactgggccacagggactacaaaattcaggcctcacgcacttggtgcttctataacactgaggatattaaagactgggaggataggttttatcttcttcttttctcattcctcggtctgctcgcactgggggtatcccttttgtgtaacgctatcacaggtatcaccttgttgcgcgttaaatttaagagccaacagcatcgccaggggaggtcacatcacctcgaaatggtaatacagttgctcgcgatcatgtgtgttagttgtatctgctggtcacccttcttgggatatagaataattctgaatggaaaagaaaagtacaaagtctatgaagaacagagcgattttctgcatcgattgcaatggcccactttggaataaa

SEQ ID NO: 55 - DNA, 1812 bp, synthetic construct
  misc_feature 1..1812: note = synthetic
  source 1..1812: mol_type = other DNA
atgcttgcccgggccctgcttctgtgtgcggtattggcactgagtcacacagccaacccgtgctgctcacacccatgtcagaatcggggagtttgcatgtcagtagggtttgaccaatataagtgcgactgtacccggacaggcttttatggagaaaattgttctacacccgaatttttgacgaggattaagctctttctgaagcctacaccaaatacagtccattatatcctcacgcacttcaagggtttttggaatgtcgtaaacaacattccgtttctgagaaatgcgataatgtcttacgtccttacatcccggagccaccttattgactcacccccaacatacaacgccgactacggatacaaaagttgggaagctttctccaacctgtcttactacacgcgagcattgcctcctgtacccgacgattgcccgacgccgttgggggtgaaaggaaaaaagcagctcccggactcaaacgaaatagtagagaaattgttgcttcgccgaaaatttataccagacccgcagggtagcaatatgatgtttgcgttcttcgctcagcacttcactcaccaattctttaagacagaccacaagagaggtcccgcattcacgaatggccttggacacggcgtcgacttgaaccacatatatggggagacattggccagacaaaggaaacttcgattgtttaaggatgggaaaatgaaataccaaattattgatggcgagatgtatcctccaacagtgaaggatacgcaagcggagatgatttatccaccgcaggtaccagagcatttgcggttcgcggtggggcaggaagtatttggacttgttcccggtctgatgatgtacgccacaatatggctccgggagcataatcgcgtgtgtgacgtacttaagcaagagcacccggaatggggcgacgagcaactttttcaaacgagtcgactgattctcattggggaaacgattaaaatagtgatcgaagattatgtgcaacatctttctggttaccactttaagttgaagttcgatcctgagctgcttttcaataaacaattccagtatcaaaacagaatcgccgcggagttcaacacgctgtatcattggcatcccctgttgcccgatacttttcaaatccatgaccagaaatacaattaccagcaatttatatataataactctatcctcctggagcatggcattacacaattcgttgaaagtttcacaaggcagatcgcaggaagagttgcgggtggccggaatgtgccgcctgccgtgcaaaaggtatcacaggctagcatcgaccaatcaaggcaaatgaagtaccagtcttttaatgagtacaggaaaaggttcatgctgaagccttacgagagctttgaagaactgacgggcgaaaaggaaatgtccgcggaactggaagcgttgtacggagatattgatgcggtagagctctaccccgcgcttctggtcgaaaagccccggccagatgccattttcggggaaaccatggttgaagtgggcgccccattcagtttgaaaggtctgatgggtaatgtaatttgcagtccggcgtactggaagccttccacttttggtggggaagtggggtttcaaattatcaatacggcctcaattcagtctctgatttgtaacaatgttaagggatgtccctttacatcttttagtgtaccggaccccgagcttattaagaccgtgaccataaatgcttcaagctctagaagtggtcttgatgatatcaaccccacagttttgcttaaggaaaggagcacggagctc

SEQ ID NO: 56 - DNA, 66 bp, synthetic construct
  misc_feature 1..66: note = synthetic
  source 1..66: mol_type = other DNA
ggaagcggagctactaacttcagcctgctgaagcaggctggagacgtcgaggagaaccctggacct

SEQ ID NO: 57 - DNA, 891 bp, synthetic construct
  misc_feature 1..891: note = synthetic
  source 1..891: mol_type = other DNA
agcatgaacaactcaaaacagttggtctccccagcagcggcccttttgtccaacacaacgtgtcagactgagaatagactcagtgtattttttagcgtgatttttatgaccgtcggtatcctgtctaactctcttgccatagcaatattgatgaaagcctatcagcgattccggcaaaagtctaaggcgtcatttttgttgttggcaagcgggcttgtcatcacggactttttcgggcacctcattaacggagccatagccgtgttcgtttatgcttcagacaaagagtggatacgattcgatcagagcaatgtgctttgcagtatctttggcatatgtatggtgtttagtggattgtgtccactcttgcttgggagcgtgatggctattgaaagatgcatcggggtgactaaacctatctttcacagtaccaaaattacatctaagcacgtcaagatgatgttgtccggggtgtgtctttttgctgtctttattgcgttgttgccaatactgggccacagggactacaaaattcaggcctcacgcacttggtgcttctataacactgaggatattaaagactgggaggataggttttatcttcttcttttctcattcctcggtctgctcgcactgggggtatcccttttgtgtaacgctatcacaggtatcaccttgttgcgcgttaaatttaagagccaacagcatcgccaggggaggtcacatcacctcgaaatggtaatacagttgctcgcgatcatgtgtgttagttgtatctgctggtcacccttcttgggatatagaataattctgaatggaaaagaaaagtacaaagtctatgaagaacagagcgattttctgcatcgattgcaatggcccactttggaataa

SEQ ID NO: 58 - DNA, 4621 bp, synthetic construct
  misc_feature 1..4621: note = synthetic
  source 1..4621: mol_type = other DNA
ggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatctaggcaactcgagaaacaaacaaactgaggtgcaggtacatccagctgacgagtcccaaataggacgaaagggagaggtgaagaatacgaccacctaggctcgaaagagcctaaaacataccttcctggattccactgctatccacaaaaagaaaaataaaaagccaccatgcttgcccgggccctgcttctgtgtgcggtattggcactgagtcacacagccaacccgtgctgctcacacccatgtcagaatcggggagtttgcatgtcagtagggtttgaccaatataagtgcgactgtacccggacaggcttttatggagaaaattgttctacacccgaatttttgacgaggattaagctctttctgaagcctacaccaaatacagtccattatatcctcacgcacttcaagggtttttggaatgtcgtaaacaacattccgtttctgagaaatgcgataatgtcttacgtccttacatcccggagccaccttattgactcacccccaacatacaacgccgactacggatacaaaagttgggaagctttctccaacctgtcttactacacgcgagcattgcctcctgtacccgacgattgcccgacgccgttgggggtgaaaggaaaaaagcagctcccggactcaaacgaaatagtagagaaattgttgcttcgccgaaaatttataccagacccgcagggtagcaatatgatgtttgcgttcttcgctcagcacttcactcaccaattctttaagacagaccacaagagaggtcccgcattcacgaatggccttggacacggcgtcgacttgaaccacatatatggggagacattggccagacaaaggaaacttcgattgtttaaggatgggaaaatgaaataccaaattattgatggcgagatgtatcctccaacagtgaaggatacgcaagcggagatgatttatccaccgcaggtaccagagcatttgcggttcgcggtggggcaggaagtatttggacttgttcccggtctgatgatgtacgccacaatatggctccgggagcataatcgcgtgtgtgacgtacttaagcaagagcacccggaatggggcgacgagcaactttttcaaacgagtcgactgattctcattggggaaacgattaaaatagtgatcgaagattatgtgcaacatctttctggttaccactttaagttgaagttcgatcctgagctgcttttcaataaacaattccagtatcaaaacagaatcgccgcggagttcaacacgctgtatcattggcatcccctgttgcccgatacttttcaaatccatgaccagaaatacaattaccagcaatttatatataataactctatcctcctggagcatggcattacacaattcgttgaaagtttcacaaggcagatcgcaggaagagttgcgggtggccggaatgtgccgcctgccgtgcaaaaggtatcacaggctagcatcgaccaatcaaggcaaatgaagtaccagtcttttaatgagtacaggaaaaggttcatgctgaagccttacgagagctttgaagaactgacgggcgaaaaggaaatgtccgcggaactggaagcgttgtacggagatattgatgcggtagagctctaccccgcgcttctggtcgaaaagccccggccagatgccattttcggggaaaccatggttgaagtgggcgccccattcagtttgaaaggtctgatgggtaatgtaatttgcagtccggcgtactggaagccttccacttttggtggggaagtggggtttcaaattatcaatacggcctcaattcagtctctgatttgtaacaatgttaagggatgtccctttacatcttttagtgtaccggaccccgagcttattaagaccgtgaccataaatgcttcaagctctagaagtggtcttgatgatatcaaccccacagttttgcttaaggaaaggagcacggagctcggaagcggagctactaacttcagcctgctgaagcaggctggagacgtcgaggagaaccctggacctagcatgaacaactcaaaacagttggtctccccagcagcggcccttttgtccaacacaacgtgtcagactgagaatagactcagtgtattttttagcgtgatttttatgaccgtcggtatcctgtctaactctcttgccatagcaatattgatgaaagcctatcagcgattccggcaaaagtctaaggcgtcatttttgttgttggcaagcgggcttgtcatcacggactttttcgggcacctcattaacggagccatagccgtgttcgtttatgcttcagacaaagagtggatacgattcgatcagagcaatgtgctttgcagtatctttggcatatgtatggtgtttagtggattgtgtccactcttgcttgggagcgtgatggctattgaaagatgcatcggggtgactaaacctatctttcacagtaccaaaattacatctaagcacgtcaagatgatgttgtccggggtgtgtctttttgctgtctttattgcgttgttgccaatactgggccacagggactacaaaattcaggcctcacgcacttggtgcttctataacactgaggatattaaagactgggaggataggttttatcttcttcttttctcattcctcggtctgctcgcactgggggtatcccttttgtgtaacgctatcacaggtatcaccttgttgcgcgttaaatttaagagccaacagcatcgccaggggaggtcacatcacctcgaaatggtaatacagttgctcgcgatcatgtgtgttagttgtatctgctggtcacccttcttgggatatagaataattctgaatggaaaagaaaagtacaaagtctatgaagaacagagcgattttctgcatcgattgcaatggcccactttggaataaaaacaaacaaactgagatgcaggtacatcccactgatgagtcccaaataggacgaaagggagaggtgaagaatacgaccacctaggctcgaaagagcctaaaacataccttctgggattccactgctatccacaaaaagaaaaataaaaagcggccgctgcaggagtcggtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacc

SEQ ID NO: 59 - DNA, 122 bp, synthetic construct
  misc_feature 1..122: note = synthetic
  source 1..122: mol_type = other DNA
ctgaggtgcaggtacatccagctgacgagtcccaaataggacgaaagggagaggtgaagaatacgaccacctaggctcgaaagagcctaaaacataccttcctggattccactgctatccac

SEQ ID NO: 60 - DNA, 1812 bp, synthetic construct; identical to SEQ ID NO: 55
  misc_feature 1..1812: note = synthetic
  source 1..1812: mol_type = other DNA

SEQ ID NO: 61 - DNA, 66 bp, synthetic construct; identical to SEQ ID NO: 56
  misc_feature 1..66: note = synthetic
  source 1..66: mol_type = other DNA

SEQ ID NO: 62 - DNA, 891 bp, synthetic construct; identical to SEQ ID NO: 57
  misc_feature 1..891: note = synthetic
  source 1..891: mol_type = other DNA

SEQ ID NO: 63 - DNA, 122 bp, synthetic construct
  misc_feature 1..122: note = synthetic
  source 1..122: mol_type = other DNA
ctgagatgcaggtacatcccactgatgagtcccaaataggacgaaagggagaggtgaagaatacgaccacctaggctcgaaagagcctaaaacataccttctgggattccactgctatccac

SEQ ID NO: 64 - DNA, 168 bp, synthetic construct; identical to SEQ ID NO: 11
  misc_feature 1..168: note = synthetic
  source 1..168: mol_type = other DNA

SEQ ID NO: 65 - DNA, 139 bp, synthetic construct; identical to SEQ ID NO: 13
  misc_feature 1..139: note = synthetic
  source 1..139: mol_type = other DNA

SEQ ID NO: 66 - DNA, 135 bp, synthetic construct; identical to SEQ ID NO: 14
  misc_feature 1..135: note = synthetic
  source 1..135: mol_type = other DNA

SEQ ID NO: 67 - DNA, 134 bp, synthetic construct; identical to SEQ ID NO: 15
  misc_feature 1..134: note = synthetic
  source 1..134: mol_type = other DNA

SEQ ID NO: 68 - DNA, 104 bp, synthetic construct
  misc_feature 1..104: note = synthetic
  source 1..104: mol_type = other DNA
gagagaatcttctttctgtctataaaagagagaatcttctttctgtctataaaagagagaatcttctttctgtctataaaagagagaatcttctttctgtctat

SEQ ID NO: 69 - DNA, 124 bp, synthetic construct
  misc_feature 1..124: note = synthetic
  source 1..124: mol_type = other DNA
gcggccgcgctagcacgggtcctgataccagcgtgagcggagagaatcttctttctgtctattagtgaagccacagatgtaatagacagaaagaagattctctccgcctacgcccttggcagca

SEQ ID NO: 70 - DNA, 21 bp, synthetic construct
  misc_feature 1..21: note = synthetic
  source 1..21: mol_type = other DNA
tatttagggaaggaactcagg

SEQ ID NO: 71 - DNA, 21 bp, synthetic construct
  misc_feature 1..21: note = synthetic
  source 1..21: mol_type = other DNA
aatacaaacctgaacaatggg

SEQ ID NO: 72 - DNA, 21 bp, synthetic construct
  misc_feature 1..21: note = synthetic
  source 1..21: mol_type = other DNA
agttgagccaagatcgacacg

SEQ ID NO: 73 - DNA, 21 bp, synthetic construct
  misc_feature 1..21: note = synthetic
  source 1..21: mol_type = other DNA
atctatactatttagggaagg

SEQ ID NO: 74 - DNA, 21 bp, synthetic construct
  misc_feature 1..21: note = synthetic
  source 1..21: mol_type = other DNA
agatcgacacgttaactcttg

SEQ ID NO: 75 - DNA, 21 bp, synthetic construct
  misc_feature 1..21: note = synthetic
  source 1..21: mol_type = other DNA
taggtacagctagatatttgc

SEQ ID NO: 76 - DNA, 21 bp, synthetic construct
  misc_feature 1..21: note = synthetic
  source 1..21: mol_type = other DNA
tcatgacgtcttaacaaacgg

SEQ ID NO: 77 - DNA, 21 bp, synthetic construct
  misc_feature 1..21: note = synthetic
  source 1..21: mol_type = other DNA
taacaaacggtaaagaaggtg

SEQ ID NO: 78 - DNA, 21 bp, synthetic construct
  misc_feature 1..21: note = synthetic
  source 1..21: mol_type = other DNA
atcaatacgagtcggaaagcg

SEQ ID NO: 79 - DNA, 21 bp, synthetic construct
  misc_feature 1..21: note = synthetic
  source 1..21: mol_type = other DNA
atctaggacactcttcgtctg

SEQ ID NO: 80 - RNA, 13 nt, synthetic construct
  misc_feature 1..13: note = Synthetic
  source 1..13: mol_type = other RNA
aaaaaaaaaaaaa

(19) United States
(12) Patent Application Publication
(10) Pub. No.: US 2024/0336925 A1
(43) Pub. Date: Oct. 10, 2024

(54) RIBOSWITCH MODULATED GENE THERAPY FOR RETINAL DISEASES

(71) Applicant: The Medical College of Wisconsin, Inc., Milwaukee, WI (US)

(72) Inventors: Daniel M. Lipinski, Milwaukee, WI (US); Chris A. Reid, Milwaukee, WI (US)

(21) Appl. No.: 18/597,673

(22) Filed: Mar. 6, 2024

Related U.S. Application Data
(63) Continuation of application No. 16/492,878, filed on Sep. 10, 2019, now Pat. No. 12,012,602.
(60) Provisional application No. 62/469,705, filed on Mar. 10, 2017.

(51) Int. Cl. C12N 15/115; A61K 48/00; C12N 15/67

(52) U.S. Cl. CPC C12N 15/115; A61K 48/005; A61K 48/0075; C12N 15/67; C12N 2310/141; C12N 2310/16; C12N 2310/3519; C12N 2320/50; C12N 2750/14143; C12N 2830/00

(57) ABSTRACT

The present invention provides constructs comprising modified riboswitches to regulate expression of a transgene within a subject. Methods of treating a disease, specifically an eye disease, are also contemplated.

### CROSS-REFERENCE TO RELATED APPLICATIONS

This Application is a continuation of U.S. application Ser. No. 16/492,878, filed Sep. 10, 2019, which is the U.S. national stage entry of PCT International Application No. PCT/US2018/021719 filed on Mar. 9, 2018, which claims priority to U.S. Provisional Application No. 62/469,705 filed on Mar. 10, 2017. The entire content of each of the above-referenced applications is incorporated by reference.

### STATEMENT REGARDING FEDERALLY SPONSORED RESEARCH

N/A

### BACKGROUND OF THE INVENTION

Age-Related Macular Degeneration (AMD) and glaucoma are leading causes of vision loss worldwide. AMD is a common eye disease among people age 50 and older. In AMD, there is damage to the macula, a small area made up of millions of light-sensing cells near the center of the retina and the part of the eye needed for sharp, central vision, and the ability to see objects that are straight ahead. Macular damage is caused by the formation of deposits called drusen, and in some cases, the growth of abnormal blood vessels, under the retina.

Glaucoma is a group of diseases in which the eye's optic nerve is damaged resulting in vision loss and blindness. The hallmark of glaucoma is increased intraocular pressure related to build-up of fluid (aqueous humor). It some patients the disease is genetic. In other patients, inflammation in the eye is thought to be involved in glaucoma.

Neither AMD nor glaucoma can be prevented. There is no treatment for early AMD in which there is no symptoms or loss of vision. Advanced AMD is treated with biologics. Glaucoma is often treated with prostaglandin eye drops. Current treatments are invasive and expensive and burdensome on the patient and clinic, requiring monthly eye injections (AMD) or daily eye drop administration.

Ocular gene therapies have the potential to profoundly improve the quality of life in patients with inherited retinal disease. Several factors make the eye an ideal organ for gene-replacement therapy. The eye is accessible, it is a compartmentalized, privileged site. This in turn means that immunologically the eyes are able to tolerate the introduction of foreign proteins/antigens without eliciting an inflammatory immune response. Clinical trials can take advantage of contralateral controls. For this reason, gene therapies for eye diseases are in development.

In this regard, recombinant adeno-associated virus (rAAV) vectors have emerged as promising tools for mediating gene therapy for diseases of the retina. Effectively controlling gene expression levels following vector delivery is paramount to the success of potential gene therapies, where uncontrolled over-expression of the therapeutic transgenes can lead to toxicity. Traditionally, inducible promoter systems have been employed. Unfortunately, due to the limited coding capacity of AAV, and the large size of the regulatory elements required to make such systems work effectively, inclusion of traditional promoters is not feasible.

Riboswitches are a possible alternative. Riboswitches are specific regulatory components of an mRNA molecule that bind and target small target molecules thereby regulating the expression of the riboswitch-containing mRNA's protein product in a cis-fashion. Thus, an mRNA that contains a riboswitch is directly involved in regulating its own activity, in response to the concentrations of its target molecule. Some riboswitches are “self-targeting”, which means that can regulate their own expression. The switch includes an RNA element that can adapt to one of two mutually exclusive secondary structures. One of these structures is a signal for gene expression to be “on” and the other conformation turns the gene “off.”

However, often toxic concentrations of ligand are necessary to “flip the switch” to turn on or off gene expression. Consequently, a goal of current research is to improve these regulatory devices toward efficiency, improved regulatory parameters, and clinical applicability. The subject of this invention are riboswitches that can turn on or off gene expression in the retina for use in ocular gene therapy.

### SUMMARY OF THE INVENTION

The present invention overcomes the aforementioned drawbacks by providing constructs, kits and methods of regulating a transgene expression using a modified riboswitch which works by a dual mechanism of gene silencing. The invention provides a distinct and improved design over prior riboswitches with its dual mechanism for gene silencing, thereby allowing for better control of expression of the transgene. Novel modified riboswitches are described in more detail herein, including a self-targeting ligand inactivating microRNA (SLIM) switch which is an ON-type switch that mediates an increase in gene expression in the presence of the ligand. Compositions and methods of using this technology to treat AMD are provided herein. Specifically, this SLIM switch can be used to intermittently switch on expression of transgene, for example, a VEGF inhibitor. Further embodiments of SLIM switches that can be used to treat glaucoma. Compositions and method of use of this technology are contemplated for the treatment of glaucoma, where it would be beneficial to intermittently turn off, or decrease expression of the prostaglandin in the eye of a patient.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic demonstrating the OFF-Switch and ON-Switch riboswitches.

FIG. 2A is a schematic representing the development of a SLIM switch (e.g. switchable miRNA) using a pri-miRNA and an aptamer.

FIG. 2B is a schematic showing the design and functioning of a SLIM switch with and without ligand.

FIG. 2C is a schematic representing the rAAV vector encoding Aflibercept (a VEGF inhibitor), with the modified switch (smiRNA) and target miRNA sequences (miRT).

FIG. 3 is a schematic of on-type aptamers that can be used in the design of SLIM switches.

FIG. 4 depicts the components of a Theo-SLIM construct.

FIG. 5 is a sequence of an exemplary construct containing Aflibercept (a VEGF inhibitor), Theo-SLIM and three miRNA target sites.

FIG. 6A is fluorescent imaging depicting GFP expression in the retina of a mouse 4 weeks post infection. C57BL6/J mouse injected intravitreally with 1.0×10¹⁰ vector genomes (vg) of rAAV2.smCBA-hGFP-3x-L2Bulge9.

FIG. 6B is fluorescent imaging of GFP two hours post-gavage @ 10 mg/kg of theophylline (activating ligand).

FIG. 7 is proposed response of using a SLIM switch (constitutively off transgene), showing monthly administration of ligand results in a spike in expression level of the transgene (e.g. aflibercept).

FIG. 8A is a schematic of the testing protocol for determining the optimal copy number of each riboswitch.

FIG. 8B is a schematic of the testing protocol for evaluation of dynamic range for each riboswitch.

FIG. 9A is a schematic representation of the mRNA of a L2Bulge18tc riboswitch construct.

FIG. 9B is a bar graph depicting the results of different copy numbers of the L2Bulge18tc riboswitch on expression levels of a transgene (GFP).

FIG. 9C is a line graph depicting the dynamic range of a construct comprising 3 copies of the L2Bulge18tc riboswitch.

FIG. 9D are fluorescent imaging of cells treated with 0-100 μM tetracycline.

FIG. 10A a schematic representation of the mRNA of a construct containing a K19 riboswitch.

FIG. 10B is a bar graph depicting the results of different copy numbers of the K19 riboswitch on transgene expression levels.

FIG. 10C is a line graph depicting the dynamic range of the optimal copy number of K19 riboswitch.

FIG. 10D are fluorescent imaging of cells treated with 0-100 μM of tetracycline which were transduced with the K19 riboswitch construct of 10A.

FIG. 11A depicts the mRNA of a construct comprising the L2Bulge9 riboswitch.

FIG. 11B is a bar graph depicting the results of different copy numbers of the L2Bulge9 riboswitch on transgene expression levels.

FIG. 11C is a line graph depicting the dynamic range of the optimal copy number of L2Bulge9 riboswitch.

FIG. 11D are fluorescent imaging of cells treated with 0-100 μM of theophylline which were transduced with the L2Bulge9 riboswitch construct of 11A.

FIG. 12A depicts the mRNA of a construct comprising the tetracycline SLIM switch (ON-type switch).

FIG. 12B depicts the dynamic range of the tet-SLIM switch of FIG. 12A.

FIG. 12C are fluorescent imaging of cells treated with 0-100 μM of tetracycline which were transduced with the tet-SLIM switch construct of 12A.

FIG. 13A depicts the mRNA of a construct comprising the theophylline SLIM switch (ON-type switch).

FIG. 13B depicts the dynamic range of the theo-SLIM switch of FIG. 13A.

FIG. 13C are fluorescent imaging of cells treated with 0-100 μM of theophylline which were transduced with the theo-SLIM switch construct of 13A.

FIGS. 14A, 14B, 14C, 14D, 14E, 14F, 14G, 14H, 141, 14J, 14K, and 14L are representative fluorescein angiography images of CNV lesions. Leakage from CNV lesions was assessed 7 days following laser injury. Representative FA images taken 5 minutes after fluorescein injection for mice injected with either (A-C) rAAV2[MAX].smCBA-Eylea, (D-F) rAAV2[MAX].smCBA-Eylca-1x-TC45+standard diet, (G-I) rAAV2[MAX].smCBA-Eylca-1x-TC45+tetracycline diet or PBS (n=16-20 lesions per group). Images of 3 mice per group with red arrows indicating site of the laser injury.

FIGS. 15A-15B demonstrate intraocular concentration of Eylea correlated strongly with severity of CNV lesions. (A) Distribution of lesions graded independently by three blinded scientists. N=16-20 lesions per group, p<0.0001, Chi-squared test). (B) Intraocular levels of non-complexed Eylea assayed by ELISA. N=5 eyes per group.

FIG. 16 are exemplary sequences of the recombinant VEGF inhibitor cDNA (SEQ ID NO:18), and complete AAV vector comprising SLIM Eylea (SEQ ID NO:19).

FIGS. 17A-17C are schematic representations of 6S-folini acid-responsive SLIM (A) (SEQ ID NO:21), theophylline-responsive SLIM (B) (SEQ ID NO:26) and tetracycline-responsive SLIM (C) (SEQ ID NO:31).

FIG. 18 are exemplary 6-S-Folinic Acid-responsive miRNA Switches (Eylea CDS target) (SEQ ID NOs:21-25), Theophylline-responsive miRNA switches (Eylea CDS target) (SEQ ID NOs:26-30), tetracycline-responsive miRNA switches (Eylea CDS target) (SEQ ID NO:31-35), and the soluble fms-like tyrosine kinase 1 (sFLT1) VEGF inhibitor cDNA (SEQ ID NO:36) that can be used with the SLIM switches to treat AMD.

FIG. 19 shows exemplary sequences for 6S-Folinic Acid-responsive miRNA SLIM Switches (sFLT1 CDS target) including SEQ ID NOs:37-41, theophylline-responsive miRNA SLIM switches (sFLT1-CDS target) including SEQ ID NOs:42-46, and tetracycline-responsive miRNA switches (sFLT1-CDS target) SEQ ID NOs:47-51.

FIG. 20 demonstrates the enzymes of the PGF2α biosynthesis pathway.

FIG. 21 is a depiction of a vector contruct of a vector (e.g. AAV vector) comprising prostaglandin endoperoxide synthase 2 (PTGS2) and PTGFR.

FIG. 22 is a graph depicting the change in intraocular pressure (IOP) from baseline in mice after treatment with an AAV with SLIM riboswitch and administration of the ligand tetracycline.

FIG. 23 are exemplary sequences for use in the present invention, including a codon optimized PTGS2 sequence (SEQ ID NO:52), a codon optimized PTGFR (SEQ ID NO:53), PTGS2-P2-PTGFR sequence (SEQ ID NO:54), and complete AAV expression cassette for PDG2alpha biosynthesis (SEQ ID NO:58). Also included are additional riboswitches that can be used in the practice of the present invention (SEQ ID NO:64-67).

### DETAILED DESCRIPTION OF THE INVENTION

The present disclosure provides modified riboswitches (e.g., modified miRNA switches) and systems containing said modified riboswitches for use in the treatment of diseases, specifically eye diseases, and more specifically AMD and glaucoma. The present invention aims to provide novel systems to regulate gene expression in the eye, using novel modified microRNA (riboswitches) as a regulatory tool. Such genetic constructs, and kits or methods utilizing same, may be of use in controlling gene expression, for example in the production of recombinant proteins or transgenes involved in the regulation of specific compounds, for example, prostaglandins in the eye.

Such RNA-based systems (riboswitches) offer three distinct advantages over inducible promoter systems. First, they have a small genetic footprint (˜100 bp) and so can be easily incorporated within the rAAV vector genome without sacrificing significant amounts of coding capacity. Second, these devices act in cis, limiting the likelihood of an immune response as no protein cofactors are required for functionality. Last, the aptamer domain of a riboswitch can be engineered to respond to almost any activating ligand, including proteins, small molecule drugs or ions.

One embodiment of this technology is to modulate expression of transgenes involved in prostaglandin F2α synthesis in the eye to regulate intraocular pressure in glaucoma patients. A second embodiment is to modulate the expression of an anti-VEGF recombinant fusion protein in the eye, namely Aflibercept (Eylea) in order to prevent choroidal neovascularization (CNV).

The technology in some embodiments uses constructs including modified riboswitches, which are ligand-controlled gene regulatory elements that allow for either switching on or off a transgene of interest in order to regulate transgene expression. The mechanisms by which riboswitches function include, without limitation, the ability to function as a ribozyme and cleave itself if a sufficient concentration of its ligand is present, the ability to fold the mRNA in such a way the ribosomal binding site is inaccessible and prevents translation from occurring, and/or the ability to affect the splicing of the pre-mRNA molecule. Embodiments comprising the modified riboswitches and constructs or cassettes containing such riboswitches are contemplated herein.

MicroRNAs are a class of non-coding RNAs that play key roles in the regulation of gene expression. Acting at the post-transcriptional level, microRNA (miRNA) genes are transcribed by RNA polymerase II as large primary transcripts (pri-mRNA) that are processed by a protein complex containing the RNase III enzyme Drosha, to form a precursor microRNA (pre-microRNA). In the present invention, the pri-microRNA have been designed to be processed into a pre-mRNA that can act as a silencing RNA to silence gene expression.

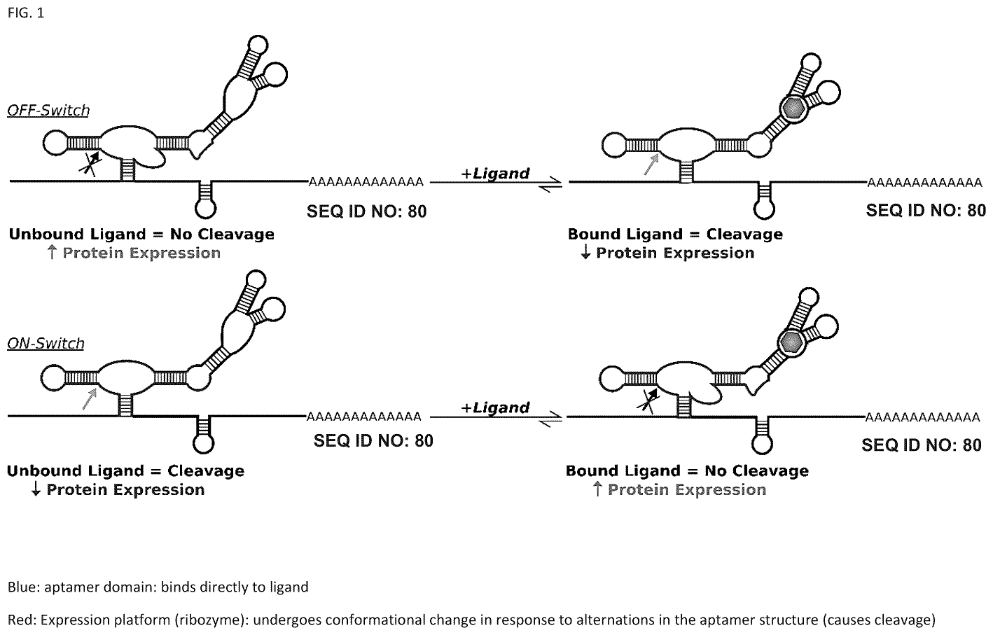

Riboswitches are divided into two parts: an aptamer and an expression platform. The aptamer is a “sensor” to which a target small molecule ligand binds; the expression platform undergoes structural changes in response to the changes in the aptamer sensor. Binding of the aptamer domain causes conformational changes within the expression platform, this conformational change regulates gene expression by turning the gene off or on. This is illustrated in FIG. 1. The aptamer is shown in blue; the expression platform, in red; the ligand in green. Often toxic concentrations of ligand are necessary to “flip the switch”. However, the present invention provides modified riboswitches that can turn on or off gene expression in the retina for use in ocular gene therapy using non-toxic levels of ligand.

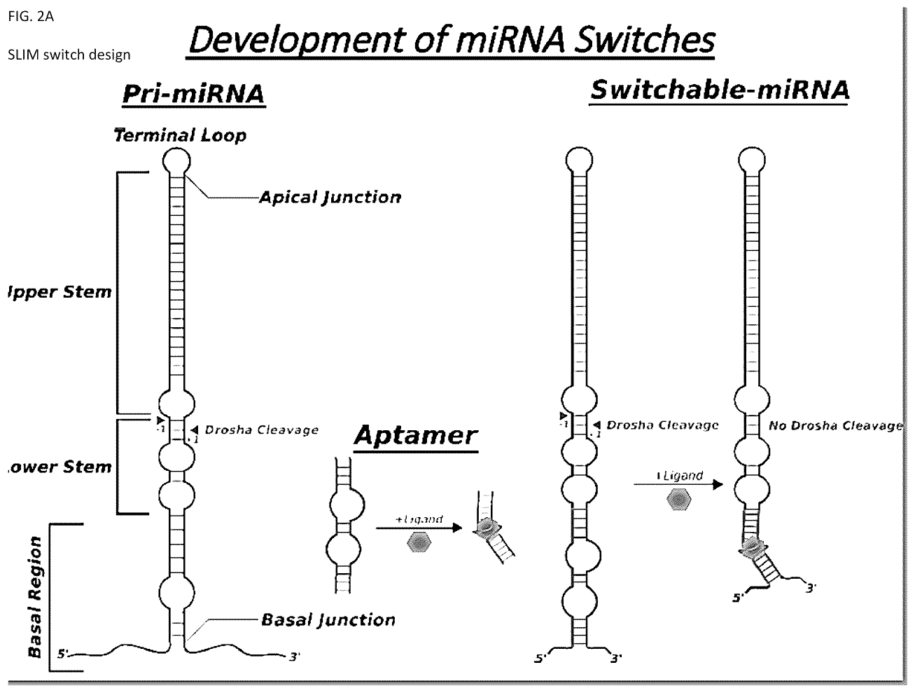

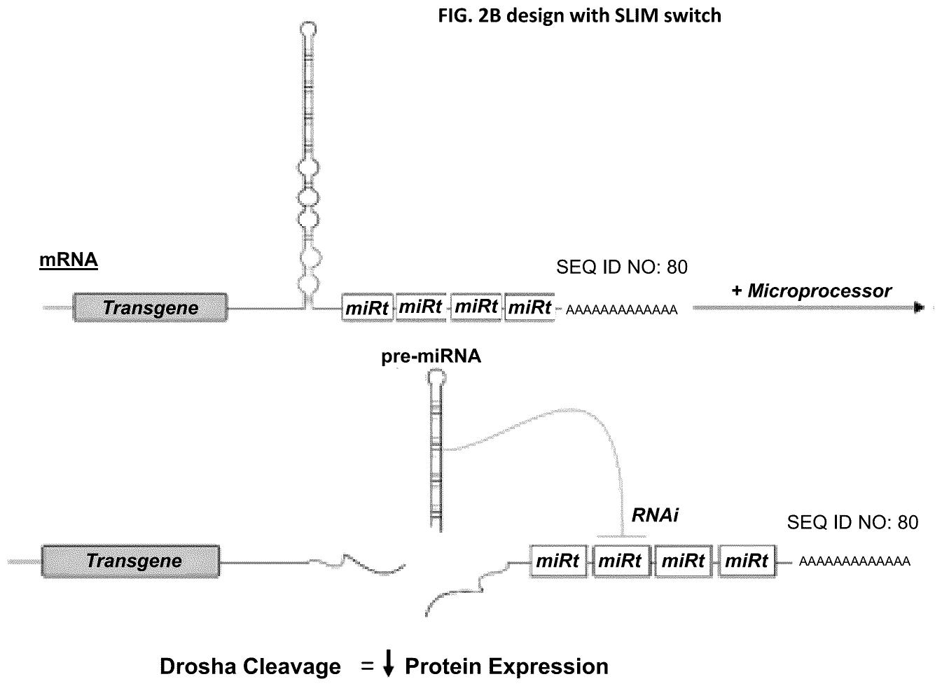

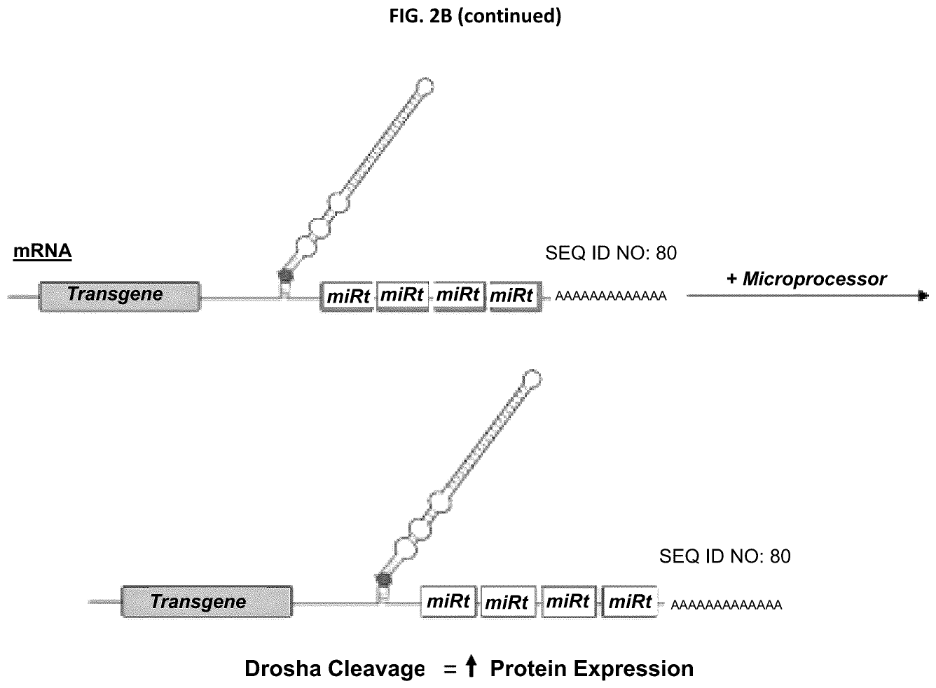

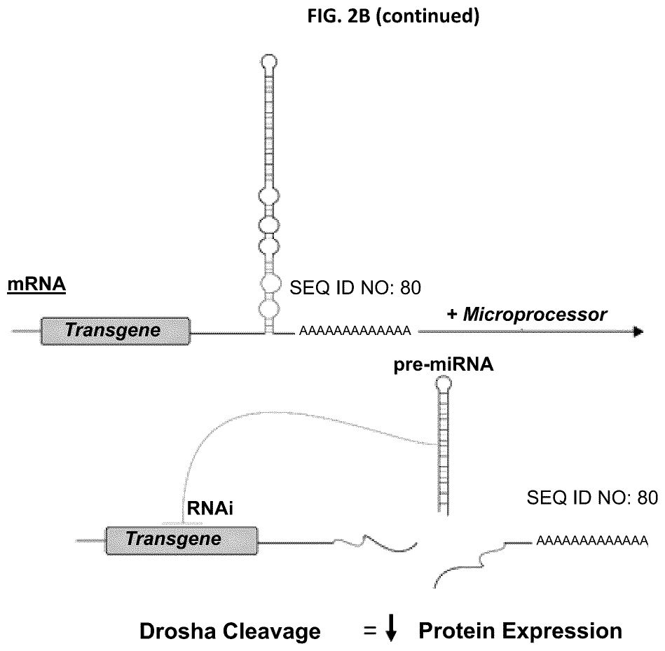

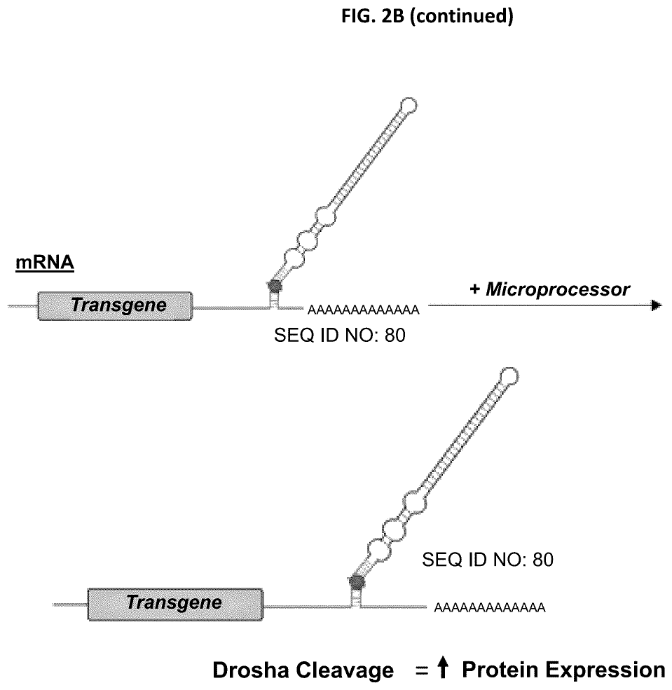

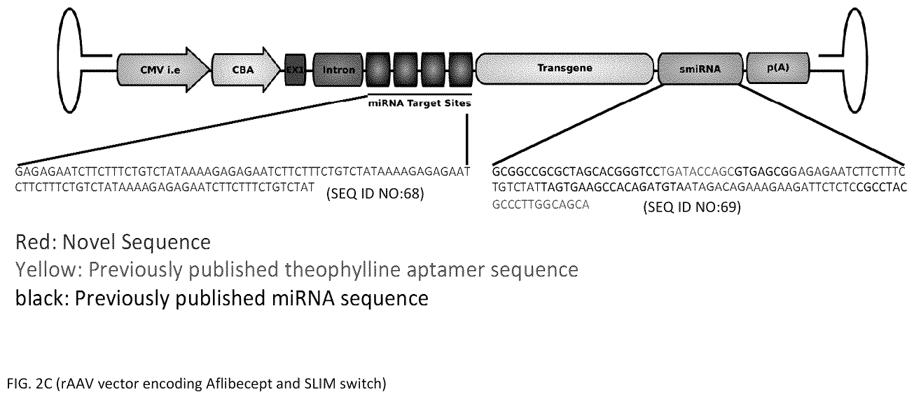

A number of exemplary riboswitches are described herein, namely a self-targeting ligand inactivating microRNA (SLIM) embodiments. A self-targeting ligand inactivating microRNA (SLIM) is an ON-type switch that mediates an increase in gene expression in the presence of the ligand (See, e.g., FIG. 2A and 2B). Compositions and methods of using this technology to treat AMD and Glaucoma are provided herein. Specifically, this SLIM system can be used to intermittently switch on expression of the VEGF inhibitor. Compositions and methods of use of this technology are contemplated for the treatment of glaucoma, where it would be beneficial to intermittently turn off or lower expression of the prostaglandin in the eye of a patient are contemplated. Both are described in more detail below.

### SLIM: ON-Type Modified Riboswitch for Modifying Gene Expression

The present invention in one embodiment provides a self-targeting ligand inactivating microRNA (SLIM) switch which is an ON type riboswitch that can be incorporated into an expression construct to regulate expression of a transgene of interest. The SLIM switch (smiRNA in FIG. 2A and B) requires the basal region of a pri-miRNA to be replaced with an aptamer that can bind to a target ligand. This sequence is cloned into either the 3′-or 5′ untranslated region of an expression construct. Binding of the ligand alters the conformation of the aptamer and pri-miRNA, resulting in non-cleavage of the pri-miRNA by Drosha. A target miRNA sequence is cloned into either the 3′ or 5′ untranslated region which allows for a second level of regulation, in which the pri-miRNA when cleaved in the absence of ligand is processed into a miRNA that can bind to the target miRNA sequence and prevent transcription. Once the SLIM switch is incorporated within an expression construct, the transgene expression is turned off when ligand is not present, and is turned on once the ligand binds to the aptamer.

SLIM switches function by regulating gene expression at the post-transcriptional level. In conditions in which the activating ligand is absent, the pri-miRNA will be cleaved by Drosha from the nascent transcript. This miRNA will be processed and act as a second mechanism of gene silencing, through binding of the complementary target sites. When the activating ligand is present, gene expression is unaltered and the gene is expressed.

In one embodiment, an exogenous nucleic acid construct for regulating expression of a transgene is provided. The construct encodes (a) a transgene, (b) a smiRNA switch (SLIM switch) located within the untranslated region of the transgene, wherein the smiRNA switch comprises an aptamer domain capable of binding to a ligand and a pri-miRNA sequence, and (c) at least one miRNA target sequence complementary to at least a portion of the pri-miRNA. The miRNA switch regulates expression of the transgene by both (1) regulation of the cleavage of the mRNA (which removes either the poly-A tail or 5′-cap destabilizing the RNA), and (2) regulating cleavage of the pri-miRNA from the smiRNA, wherein at least a portion of the cleaved pri-miRNA is processed and binds to the at least one miRNA target sequences silencing the transgene expression.

As discussed above, in the absence of ligand, the pri-miRNA is cleaved from the transcript and binds to the at least one miRNA target sequence which silences the transgene. This is depicted in FIG. 2B. In some embodiments, the at least one miRNA target sequence complementary to at least a portion of the pri-miRNA (c) discussed above is encompassed in the transgene (as depicted in the bottom figure of FIG. 2B). In other words, the SLIM switch can be generated toward the transgene itself.

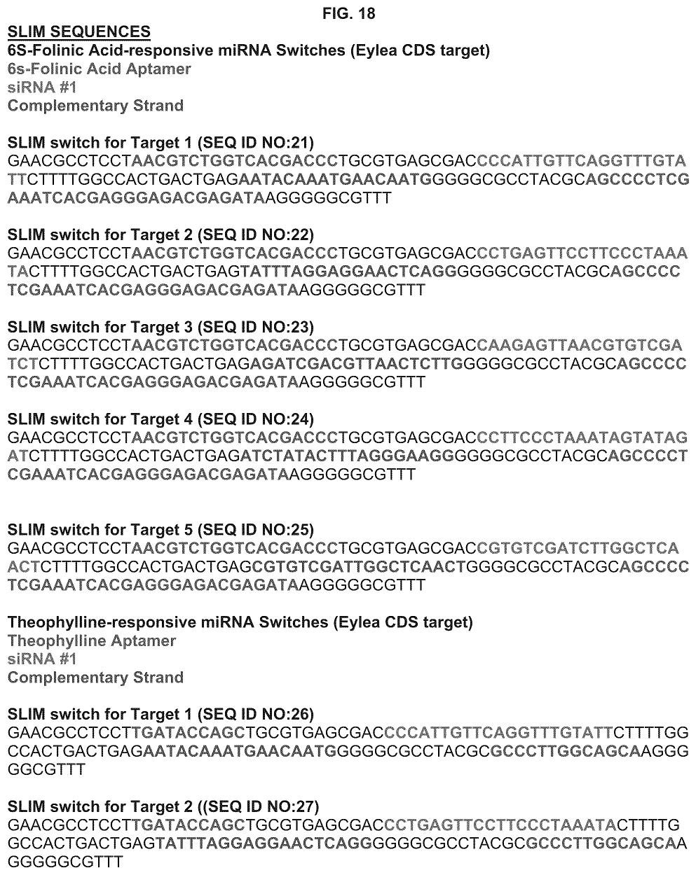

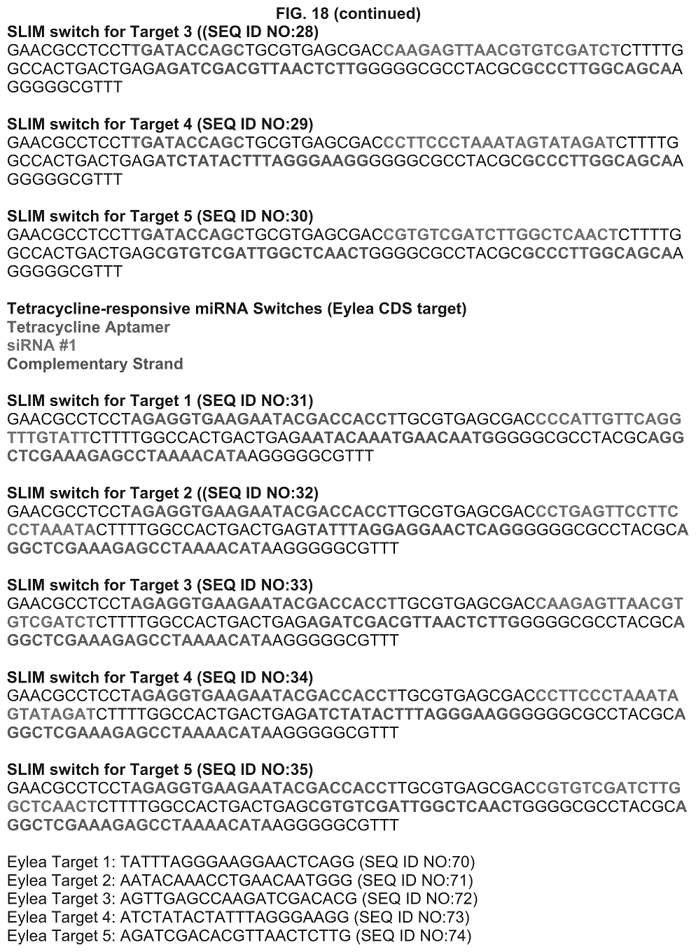

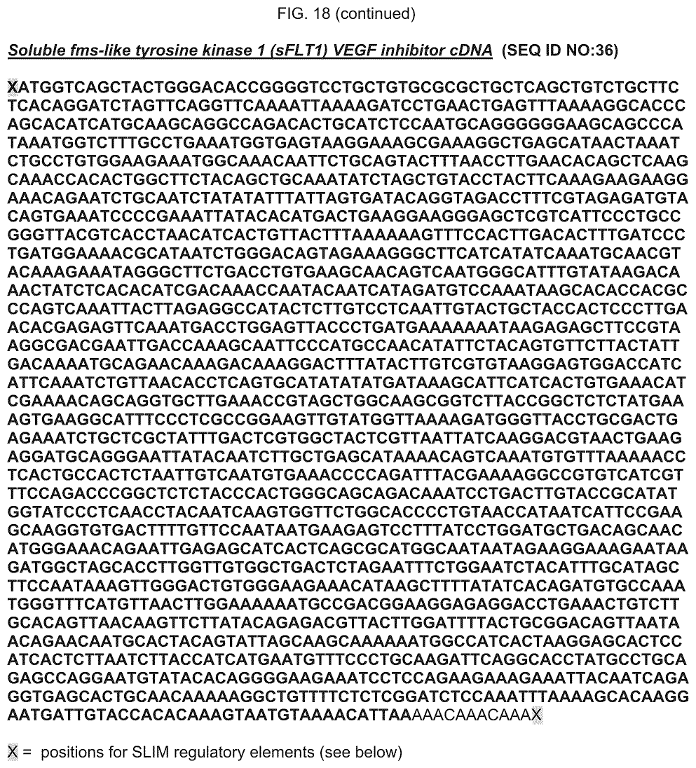

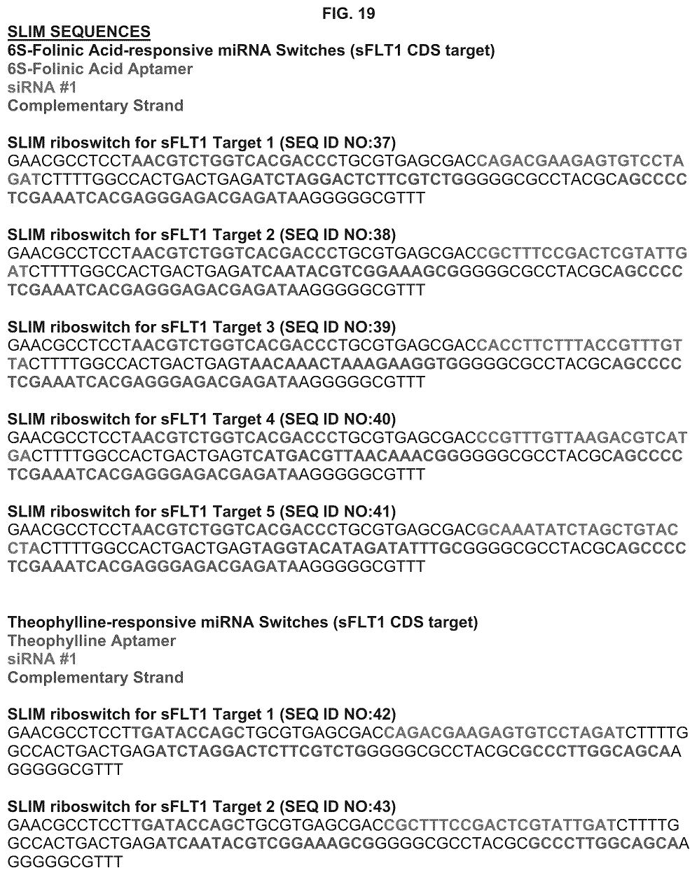

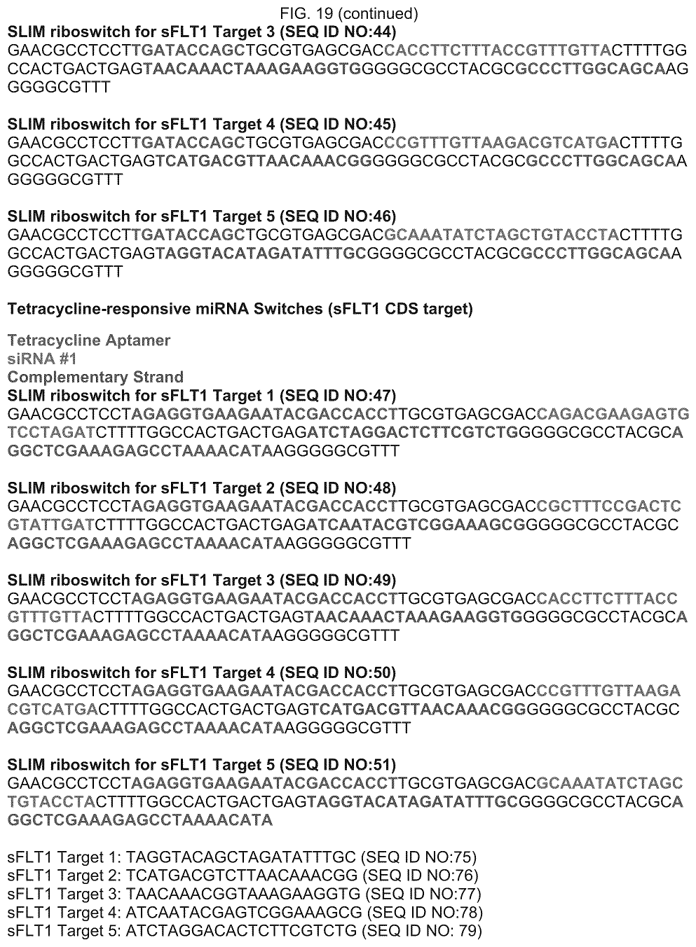

In a preferred embodiment, the heterologous self-targeting ligand inactivating microRNA (SLIM) switch comprises (a) a target gene of interest, (b) at least one smiRNA switch located within the untranslated region of the transgene, wherein the smiRNA switch comprises an aptamer domain capable of binding to a ligand and a pri-miRNA, wherein the smiRNA switch regulates expression of the transgene by both (1) regulation of the cleavage of the mRNA of the transgene, and (b) regulating cleavage of the pri-miRNA from the smiRNA, wherein at least a portion of the cleaved pri-miRNA binds to a portion of the transgene (in other words, a portion of the transgene acts as the miRNA targeting sequence) silencing the transgene expression. Suitable riboswitches are described in FIGS. 18 and 19, which also include the portion of the Eylea transgene or Flt1 transgene each riboswitch targets (e.g. SLIM switch for Eylea Target 1 (e.g., SEQ ID NO: 21, or 26 or 31) targets a portion of Eylea found in Eylea target 1 (SEQ ID NO:70), SLIM switch for Target 2 (e.g. SEQ ID NO:22, 27, 32) target Eylca Target 2 (SEQ ID NO:71) and so forth (e.g. SLIM switch for Eylea 3 targets Eylea Target 3, SLIM switch for Eylea 4 targets Eylea Target 4, SLIM switch for Eylea Target 5 targets Eylea Target 5, SLIM switch for Flt1 Target 1 targets Flt1 Target 1, etc.). One or more SLIM switches can be combined within the exogenous expression constructs for use in the methods described herein (e.g., SLIM switch for Target 1, Target 2, Target 3, Target 4, and Target 5 of Elyca or Sflt1 can be combined in any combination for a construct to target (e.g. SLIM 1 and 2; SLIM 1 and 3; SLIM 1 and 4; SLIM 1 and 5; SLIM 2 and 3; SLIM 2 and 4; SLIM 2 and 5; SLIM 3 and 4; SLIM 3 and 5; SLIM 4 and 5; SLIM 1, 2, and 3; SLIM 1, 2 and 4; SLIM 1, 2 and 5; SLIM 1, 3, and 4; SLIM 1, 3 and 5; SLIM 1, 4 and 5; SLIM 2, 3 and 4; SLIM 2, 3 and 5; SLIM 2, 4 and 5; SLIM 3, 4 and 5; SLIM 1, 2, 3, and 4; SLIM 1, 2, 3 and 5; SLIM 2, 3, 4 and 5; and SLIM 1, 2, 3, 4 and 5) of the SLIM found in FIGS. 18-19.

In one embodiment, an exogenous nucleic acid construct for regulating expression of a transgene by modulating the mRNA of the transgene is provided. The nucleic acid encoding: (a) a target gene of interest, (b) at least one smiRNA switch located within the untranslated region of the transgene, wherein the smiRNA switch comprises an aptamer domain capable of binding to a ligand and a pri-miRNA, wherein the smiRNA switch regulates expression of the transgene by both (1) regulation of the cleavage of the mRNA of the transgene, and (b) regulating cleavage of the pri-miRNA from the smiRNA, wherein at least a portion of the cleaved pri-miRNA binds to a portion of the transgene (as a miRNA targeting sequence) silencing the transgene expression. In a preferred embodiment, the exogenous nucleic acid construct is a AAV viral vector.

In some embodiments, the SLIM switch generated against Eylea or sFlt1 are used for the treatment of AMD as shown in the sequences described in FIGS. 18 and 19.

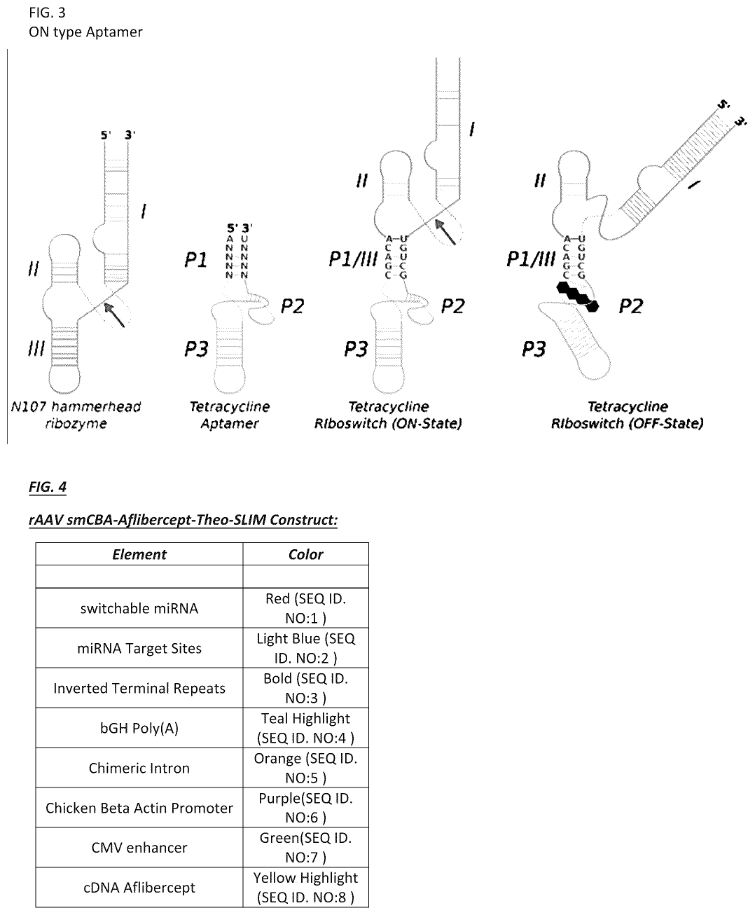

In one embodiment, an exogenous nucleic acid construct encodes: the SLIM switch, transgene, and at least one target miRNA sequence. Design of such SLIM switches is depicted in FIG. 2A. In one example, a suitable SLIM switch (smiRNA) encodes an aptamer domain and a pri-miRNA. Suitable aptamer domain is adapted from the aptamer domain of an ON-type riboswitch known in the art, and include but are not limited to, for example, L2Bulge18tc (for example, but not limited to, SEQ ID NO:12), K19 (For example, SEQ ID NO:15), L2Bulge9 (for example, SEQ ID NO:11), among others. Suitable aptamer domains are depicted in FIG. 3 and described in the specification below.

Suitable SLIM switches include, but are not limited to our developed Theo-SLIM (SEQ ID NO: 1) which is depicted in FIG. 3 (smiRNA), and Tet-SLIM (SEQ ID NO:17) as described herein. These SLIM switches have been engineered as to not cross-react with any genes in the human genome, and thus should not cause any off-target effects.

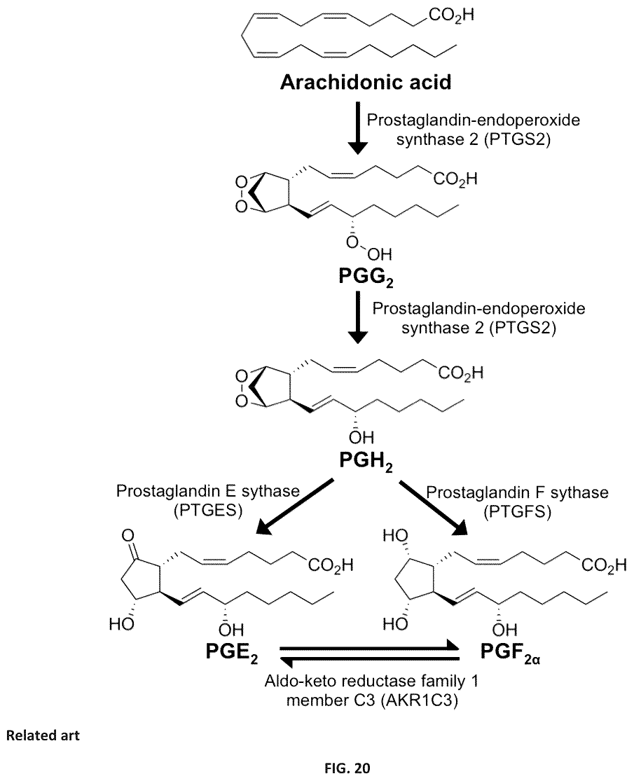

Additional SLIM switches are depicted in FIGS. 18-23.

The term “exogenous” as it refers to nucleic acid is foreign to (not normally found in nature in) the prokaryotic host cell, or a recombinant nucleic acid that is not normally found in the prokaryotic host cell. In some embodiments, the exogenous nucleic acid sequence is a heterologous sequence comprising sequences from a number of different sources or organisms. The term exogenous also encompasses a construct that include sequences from a different species than the species the construct will be used. For example, a rAAV construct of the present invention will include not only endogenous AAV sequences, but exogenous sequences from human or other sources that are not native to the AAV virus.

The nucleic acid constructs provided herein are synthetic engineered constructions containing non-naturally occurring sequences.

The term “transgene” and “target gene of interest” are used interchangeably to refer to an exogenous gene which is to be expressed in the desired cell by use of the constructs of the present invention.

Further, suitable aptamers can be designed against various ligands and incorporated into the SLIM switches by linking the aptamer to a pri-miRNA sequence (See, e.g., FIG. 2A).

In some embodiments, the constructs comprise a transgene and a SLIM switch. In some of these embodiments, the transgene acts as the miRNA target sequence for the SLIM switch. In other embodiments, the constructs further comprises the SLIM switch which also contain at least one target miRNA sequence, preferably from about 1-4 target miRNA sequences in the 3′ or 5′ UTR. For example, FIG. 2B (top figure) shows a suitable design of such construct. Suitable ligands are discussed more below, and specifically include ligands that are able to cross the blood retinal barrier, for example, tetracycline, theophylline and guanine.

In some embodiments, the construct comprises at least one target miRNA sequence, alternatively at least two, alternatively at least three, alternatively at least 4 target miRNA sequences. The number of target miRNA sequences added may depend on the vector used, for example, in the use of a rAAV vector, a suitably number of target miRNA include, for example, from 1-5. Adding more target sites would limit the available coding sequences as there is a limit to the size of the rAAV vectors, thus depending on the size and sequence of the transgene may also alter the number of target miRNA sequences added.

In one embodiment, when the smiRNA is SEQ. ID NO.1, the target miRNA sequence is encoded in SEQ ID NO: 10 (GAGAGAATCTTCTTTCTGTCTATAAAA). In one suitable embodiment, the construct contains at least three target miRNA sequence, for example, as found in SEQ ID NO: 2.

In one embodiment, the construct comprises a suitable transgene in which the expression of the transgene can be induced by administration of the ligand. For example, as described in more detail below, the transgene may be a VEGF inhibitor, specifically a VEGF inhibitor which can be induced to be expressed in the eye for the treatment of AMD. Specifically, a suitable VEGF inhibitor is aflibercept, which is encoded by the cDNA found in SEQ ID NO: 8.

In another embodiment, the aptamer domain and the pri-miRNA are encoded within SEQ ID NO:1. In some embodiments, the construct contains other sequences found within constructs, for example, promoters, enhancers, WPRE elements, and the like. One skilled in the art would be able to incorporate other known elements necessary for transgene expression from a nucleic acid construct.

In some embodiments, the construct is an adeno-associated virus (rAAV), a lentivirus, an adnovirus, a plasmid, a herpes simplex virus, a baculovirus, a bacteriophage, among others. Preferably, the construct is an adeno-associated virus. A suitable rAAV construct is demonstrated in FIG. 2 which incorporates the transgene for aflibercept, a Theo-SLIM switch and 3 miRNA target sequences, for example, the sequence encoded in SEQ ID NO:9 or SEQ ID NO:19.

In some embodiments, the SLIM switch requires the basal region of a pri-miRNA to be replaced with an aptamer. This sequence is cloned into either the 3′-or 5′ untranslated region of an expression cassette. Additionally, miRNA target sites that are complementary to the sequence of the mature miRNA can be included in one or multiple copies at either the 5′ or 3′ untranslated region of the cassette.

Self-targeting Ligand Inactivated miRNAs (SLIM) switches function by regulating gene expression at the post-transcriptional level. In conditions when the activating ligand is absent, the pri-miRNA will be cleaved by drosha from the nascent transcript. This miRNA will be processed and act as the second mechanism of gene silencing, through binding of the complementary target sites. When the activating ligand is provided, gene expression is unaltered.

Multiple copies of each riboswitch can be included into the 3′-untranslated region of the gene of interest. Each riboswitch appears to have an optimal number of copies as shown in Table 1. Furthermore, in some embodiments, multiple copies of miRNA target sites can be included in the 3′ or 5′ untranslated region. The synthetic riboswitches comprise an aptamer and an expression platform and rely on changes in the expression platform activity for regulating gene expression. The miRNA target sites are sequences in the therapeutic cassette (construct) that are recognized by the mature miRNA/siRNA. As discussed more below, the miRNA target sites may be encompassed within the transgene. In other words, the cleaved and processed siRNA from the riboswitch binds to the transgene and inhibits its expression.

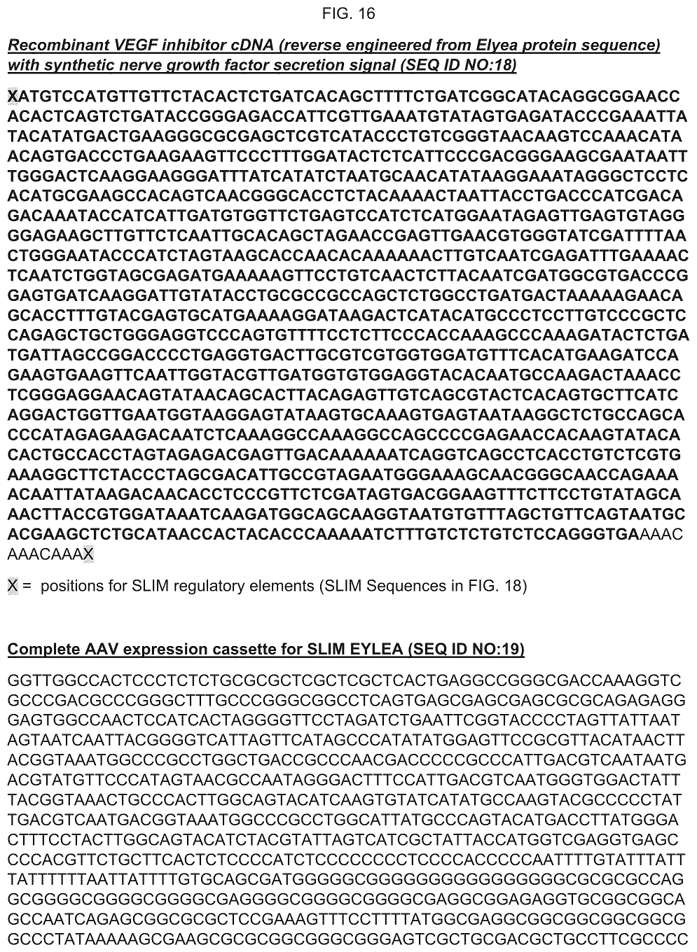

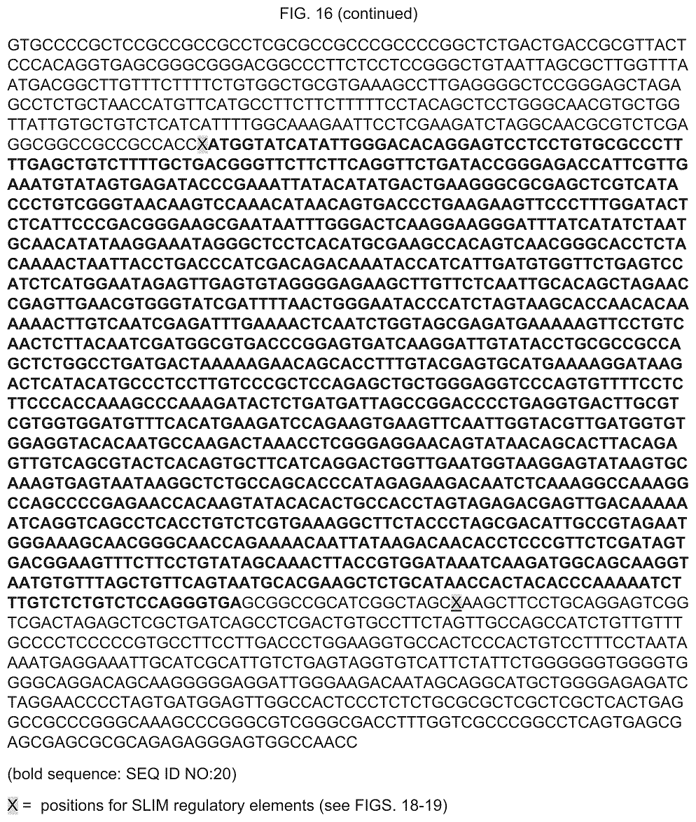

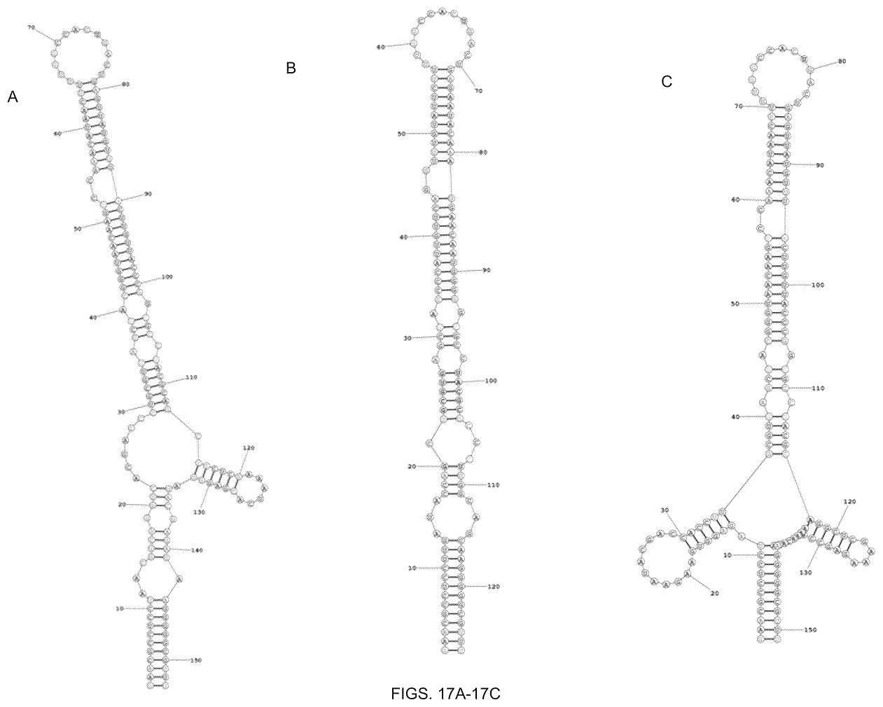

Alternatively, the SLIM switch can be generated towards the transgene itself, with Eylea and sFlt1 for the treatment of AMD, the sequences are found in FIGS. 16-18, 19 and 23.

In one embodiment, the nucleic acid construct comprises a target gene of interest, for example, a VEGF inhibitor (e.g. Eylea (SEQ ID NO:20) or sFLT1 (SEQ ID NO:36) and at least one miRNA SLIM switch located within the untranslated region of the target gene (e.g. at least one selected from SEQ ID NO:21-35 or SEQ ID NO:37-51, respectively of the VEGF inhibitors).

In some embodiments, the nucleic acid construct is an rAAV vector comprising the Eylea gene (e.g. SEQ ID NO:19). For example, SEQ ID NO:19 provides a complete AAV expression vector for the expression of Eylea using a SLIM switch. Within SEQ ID NO:19, X marks positions in which a SLIM sequence may be inserted. Suitable SLIM sequences that may be inserted can be found in FIG. 18, and include, but are not limited to, 6S-Folinic Acid-responsive SLIM (miRNA) switches, including, for example, SEQ ID NO: 21, 22, 23, 24, and 25 (activated by the ligand 6S-Folinic acid), Theophylline-responsive SLIM, for example, SEQ ID NO:26, 27, 28, 29 and 30 (activated by ligand theophylline), or Tetracycline-responsive SLIM, for example, SEQ ID NO:31, 32, 33, 34, and 35 (activated by ligand tetracycline). In some embodiments, from 1-5 SLIM sequences are inserted within the AAV expression vector, alternatively from 1-3 SLIM sequences. For example, SEQ ID NO:19 may incorporate 1-5 copies of SEQ ID NO:21, alternatively 1-5 copies of SEQ ID NO:22, alternatively 1-5 copies of SEQ ID NO: 23, alternatively 1-5 copies of SEQ ID NO:24, alternatively 1-5 copies of SEQ ID NO:25, alternatively 1-5 copies of SEQ ID NO:26, alternatively 1-5 copies of SEQ ID NO:27, alternatively 1-5 copies of SEQ ID NO:28, alternatively 1-5 copies of SEQ ID NO:29, alternatively 1-5 copies of SEQ ID NO:30, alternatively 1-5 copies of SEQ ID NO:31, alternatively 1-5 copies of SEQ ID NO:32, alternatively 1-5 copies of SEQ ID NO:33, alternatively 1-5 copies of SEQ ID NO:34, alternatively 1-5 copies of SEQ ID NO:35.

In another embodiment, the nucleic acid construct comprises a target gene of interest, for example, a VEGF inhibitor sFLT1 (SEQ ID NO:36) and at least one smiRNA SLIM switch located within the untranslated region of the target gene (e.g. at least one selected from SEQ ID NO:37-51).

In some embodiments, the nucleic acid construct comprises the sFLT1 gene (SEQ ID NO:36). sFLT1 may be inserted within a vector, for example a rAAV vector, and flanked by X which marks positions in which a SLIM sequence may be inserted. Suitable SLIM sequences that may be inserted can be found in FIG. 18, and include, but are not limited to, 6S-Folinic Acid-responsive SLIM (miRNA) switches, including, for example, SEQ ID NO: 37, 38, 39, 40, 41 (activated by the ligand 6S-Folinic acid), Theophylline-responsive SLIM, for example, SEQ ID NO:42, 43, 44, 45, 46 (activated by ligand theophylline), or Tetracycline-responsive SLIM, for example, SEQ ID NO:47, 48, 49, 50, and 51 (activated by ligand tetracycline). In some embodiments, from 1-5 SLIM sequences are inserted within the construct, alternatively from 1-3 SLIM sequences. For example, SEQ ID NO:36 may incorporate as X from 1-5 copies of SEQ ID NO:37, alternatively from 1-5 copies of SEQ ID NO:38, alternatively from 1-5 copies of SEQ ID NO:39, alternatively from 1-5 copies of SEQ ID NO:40, alternatively from 1-5 copies of SEQ ID NO:41, alternatively from 1-5 copies of SEQ ID NO:42, alternatively from 1-5 copies of SEQ ID NO:43, alternatively from 1-5 copies of SEQ ID NO:44, alternatively from 1-5 copies of SEQ ID NO:45, alternatively from 1-5 copies of SEQ ID NO:46, alternatively from 1-5 copies of SEQ ID NO:47, alternatively from 1-5 copies of SEQ ID NO:48, alternatively from 1-5 copies of SEQ ID NO:49, alternatively from 1-5 copies of SEQ ID NO:50, or alternatively from 1-5 copies of SEQ ID NO:51. Alternative combinations of SLIM sequences are contemplated (e.g. for example from 1-5 SLIM sequences selected from SEQ ID NO:36, 37, 38, 39, 40, and 41). Suitable, one would preferably use SLIM sequences activated by the same ligand in the construct, for example 1-5 SLIM sequences that are active by the tetracycline ligand.

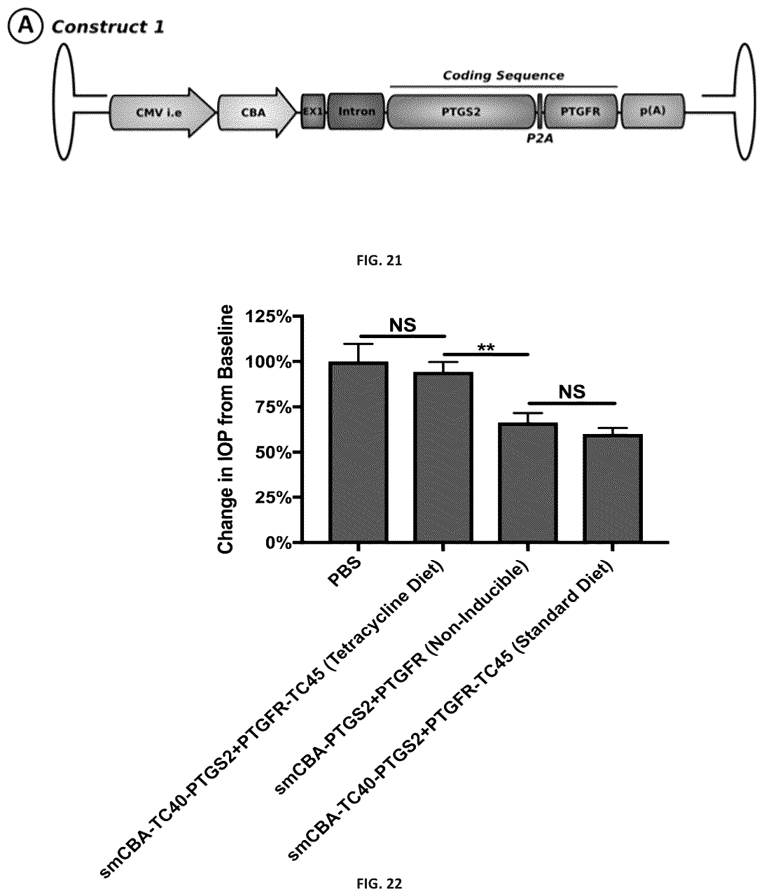

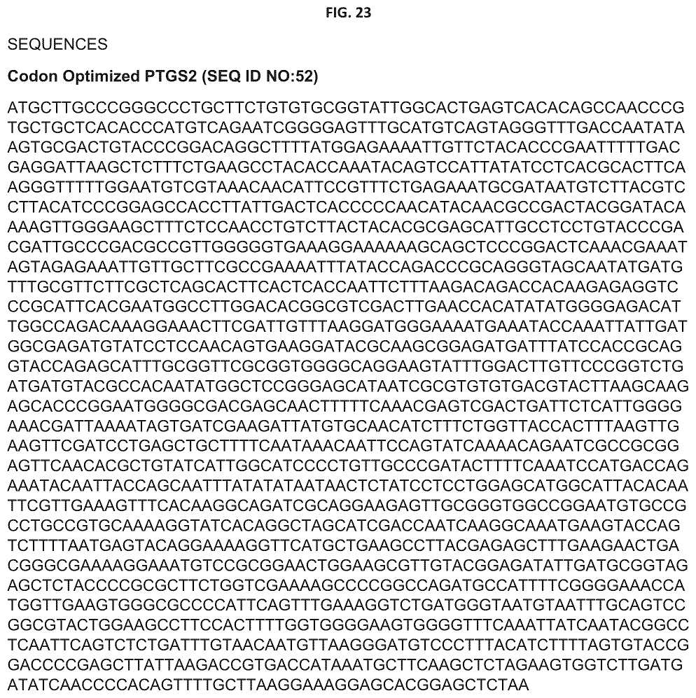

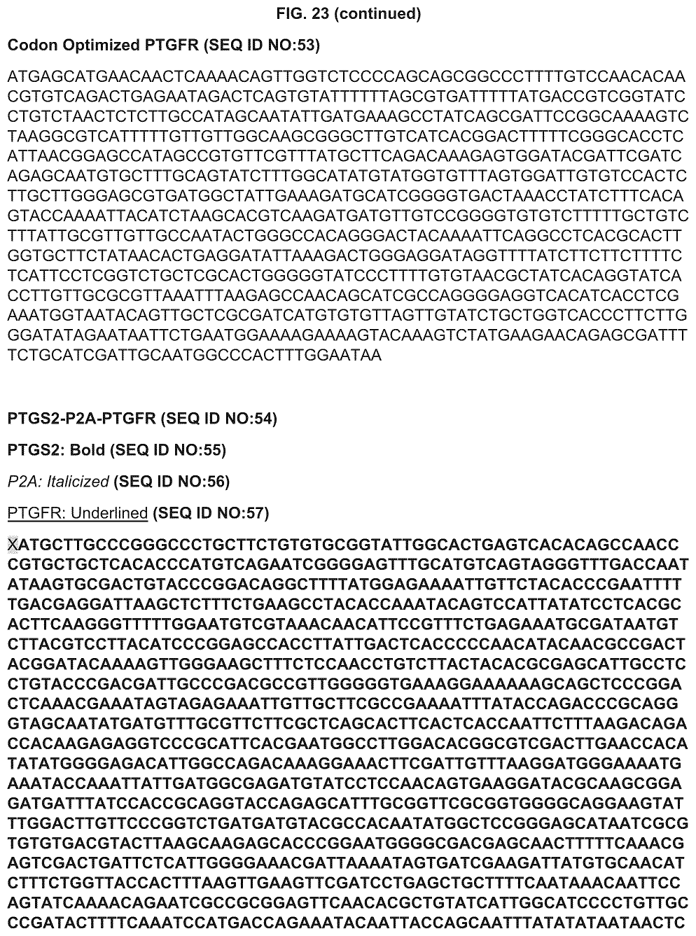

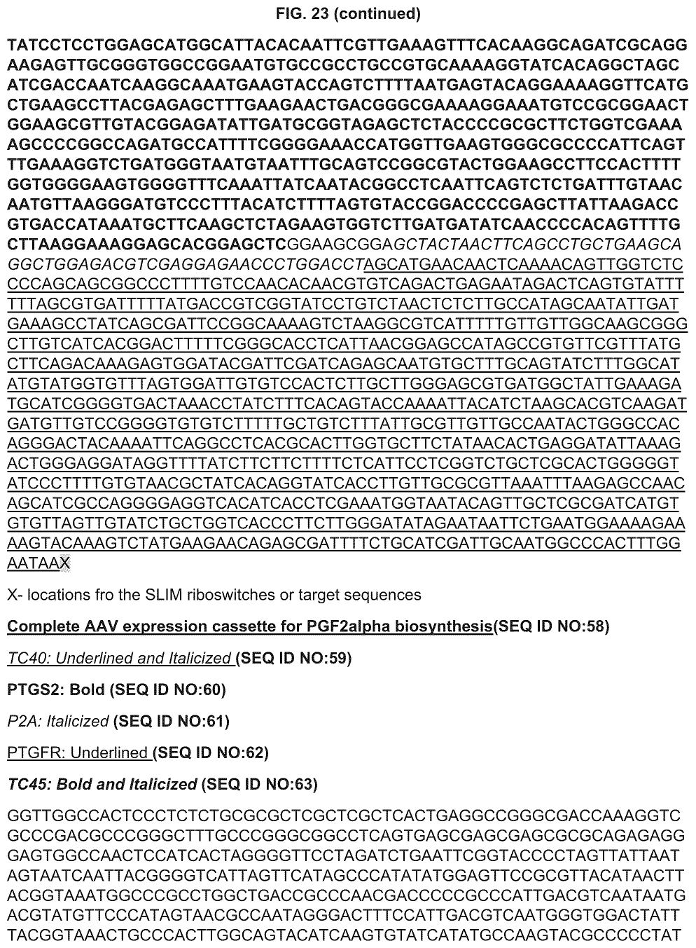

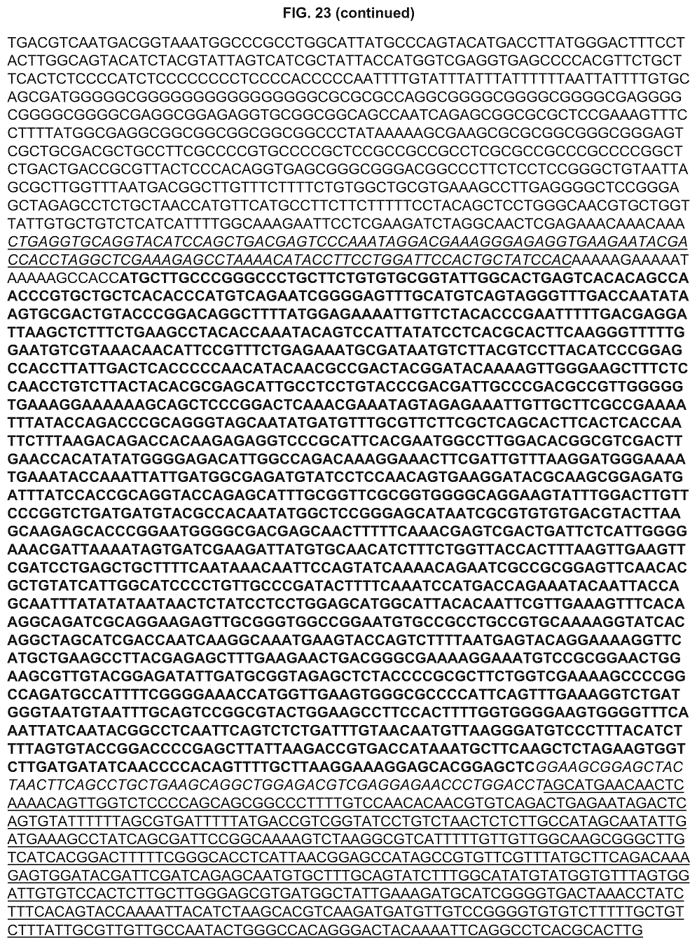

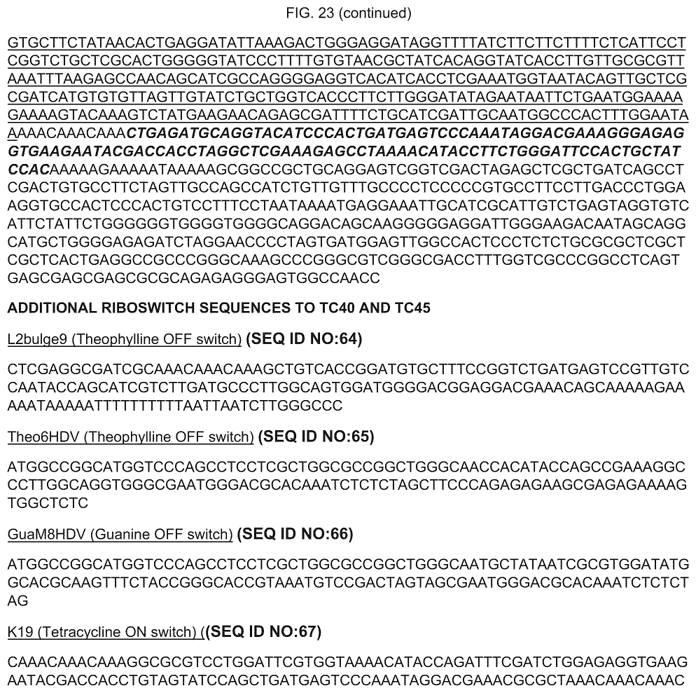

In another embodiment depicted in FIG. 21, a nucleic acid construct comprises PTGS2 (SEQ ID NO:52) and PTGFR (SEQ ID NO:53) (depicted in combination in SEQ ID NO:54 as PTGS2-P2A-PTGFR). Suitable nucleic acid construct is depicted in FIG. 23 as SEQ ID NO:81, a AAV expression vector comprising PGF2alpha biosynthesis regulatory genes (PTGS2 (SEQ ID NO:60) and PTGFR (SEQ ID NO:62) including two synthetic riboswitches TC40 (SEQ ID NO:59) and TC45 (SEQ ID NO:63). Suitable use of such construct is for the treatment of glaucoma.

### Ligands

The specific ligand to be used depends on the specific application and aptamer incorporated into the SLIM switches. For the treatment of eye diseases and disorders, ligands that are able to cross the blood retinal barrier are preferred. Suitable aptamers that can cross the blood retinal barrier include, but are not limited to, for example, tetracycline, theophylline, guanine, galactitol, progesterone, mannitol, estradiol, dopamine, quinidine, urea, digoxin, uracil, verapamil, thiourea, moxifloxacin, thymine, levofloxin, corticosterone, acetazolamide, testosterone, doxycycline, and combinations thereof.

In a preferred embodiment, the ligand is selected from the group consisting of tetracycline, theophylline and guanine.

Suitable routes of administration of the ligands are known in the art and include, oral administering, administering via the eye (e.g., eyedrops) and the like.

### Treatment of Eye Diseases

The constructs of the present technology are particularly useful for gene therapy for the treatment of eye diseases. The eye is a particularly good target for this type of gene therapy for a number of reasons. The eye is a highly specialized organ which has evolved to transduce light stimuli into electrical signals and to relay those signals to the visual cortex. Light sensation and image formation is mediated through the activation of photoreceptor cells located in the outermost layer of the neurosensory retina, where incident light focused by the cornea and lens results in the activation of a signaling cascade and the propagation of an electrical impulse. Despite its complexity the eye has many traits which make it an attractive organ for gene therapy: it is relatively immune privileged, has a small compartment size, is easily visualized and examined, and readily accessible with minimal risk to patients undergoing surgery. The retina (AMD) or the cornea (Glaucoma) are the primary targets for gene therapy treatments. Vector delivery is usually achieved through injection of a fluid suspension containing the therapeutic particles into the anatomically constrained space adjacent to the target cells. A rAAV.SLIM.αVEGF or rAAV.SLIM.sFLT1 vector would be most beneficial if targeted towards cells of the inner retina, including retinal ganglion cells and Müller glia, and as such would be administered via intravitreal injection. The rAAV.SLIM.PTGS2-P2-PTGFR vector would be most beneficial if targeted towards cells of the cornea and anterior chamber, including corneal endothelial cells, and as such would be administered via intracameral injection. Numerous rAAV clinical trials using rAAV for ocular gene therapy applications have been successfully completed; however, all trials to date have utilized a sub-retinal delivery approach designed to target photoreceptors. A current phase I/II clinical trial (NCT01494805) is underway utilizing rAAV-mediated overexpression of soluble fms-like tyrosine kinase-1 (sFLT), a soluble receptor of VEGF, for the treatment of AMD. Expression of sFLT in this trial is constitutive, and cannot be regulated, unlike the proposed rAAV.SLIM.αVEGF and rAAV.SLIM.sFLT1 technology described herein. No rAAV-based gene therapy clinical trials are underway for the treatment of glaucoma.

### Treatment of AMD

The present invention provides methods of treating age-related macular degeneration by inhibiting, reducing or alleviating at least one symptom of AMD. AMD is a disease involving multiple tissue layers within the eye, including the choroid, retinal pigment epithelium and the neurosensory retina, and occurs in two forms. Dry AMD is a non-proliferative disease state characterized by progressive geographic atrophy of the central retina. In approximately 10-15% of patients the disease progresses to a wet form, characterized by abnormal growth of blood vessels from the choroid into the subretinal space. Choroidal neovascularization (CNV) results primarily as a result of increased intraocular concentrations of VEGF and is the major vision-threatening symptom of AMD. Administration of anti-VEGF agents is known to significantly reduce the incidence of CNV and is the current gold-standard treatment AMD. The treatment regimen is invasive, however, requiring repetitive (e.g. monthly or bimonthly) intravitreal injection of purified recombinant anti-VEGF protein.

The proposed rAAV.SLIM.αVEGF technology would act to significantly reduce the incidence of CNV formation in AMD patients, and consequently prevent vision loss. Critically, the inclusion of the SLIM technology would allow for anti-VEGF expression to be controlled through dosing of the activating ligand.

The rAAV.SLIM.αVEGF vector technology of the present invention would act to significantly reduce the incidence of CNV formation in AMD patients, and consequently prevent vision loss. Critically, the inclusion of the SLIM technology would allow for anti-VEGF expression to be controlled through dosing of the activating ligand.

The present invention provides methods of treating, reducing, alleviating or inhibiting at least one symptom of AMD comprising administering to the eye of a subject a construct comprising a VEGF inhibitor and a SLIM riboswitch as described herein (including, e.g., rAAV.SLIM.αVEGF or rAAV.SLIM.sFLT1 vector), and further administering a therapeutically effective amount of the ligand. The SLIM riboswitch can determine which ligand is used as described in more detail herein. Suitably, the treatment results in the reduction, alleviation or inhibition of one or more symptom of AMD, for example, a reduction or inhibition of the development of CNV.

In one embodiment, the method of treating, inhibiting or reducing at least one symptom of AMD comprises administering a construct comprising one or more ON-type riboswitch operably connected to a transgene encoding a VEGF inhibitor and administering a therapeutically effective amount of the ligand. Suitable ON-type riboswitches are known in the art and include, but are not limited to, for example, L2Bulge18tc (SEQ ID NO:12), K19 (SEQ ID NO:15), and L2Bulge 9 (SEQ ID NO:11). Further, the construct may encode an optimal copy number of the ON-type riboswitches which can be incorporated into the construct. The optimal copy number of the riboswitch is detailed in Table. 1. For example a construct for the treatment of AMD may comprise from 1-3 L2Bulge18tc riboswitches and a VEGF inhibitor.

TABLE 1
ON-TYPE Switches and SLIM switches

| | Activating | Optimal | | Dynamic | SEQ |
|---|---|---|---|---|---|
| Riboswitches | Ligand | Copy number | | Range | ID NO: |
| L2Bulge18tc | Tetracycline | 3 | 12.5 | 4.3-fold | 12 |
| K19 | Tetracycline | 1 | 39.1 | 1.5-fold | 15 |
| L2Bulge9 | Theophylline | 3 | 37.9 | 1.7-fold | 11 |
| Theo-SLIM | Theophylline | 1 | 7.2 | 10.9-fold | 1 |
| Tet-SLIM | Tetracycline | 1 | 36.4 | 2.3-fold | 17 |

The SLIM riboswitches can be incorporated into constructs of the present invention to alter gene expression of the transgene by administration of a therapeutically effective amount of the ligand. By “therapeutically effective amount” we mean an amount or dosage of the ligand that is able to alter the expression level of the transgene product in a cell. One skilled in the art will be able to titrate and determine a therapeutically effective amount to produce the proper response and result in a reduction of the symptoms of the desired disease to be treated. A therapeutically effective amount also maintains that the level of ligand is in a non-toxic dose to the subject. Suitably, the dosage may be given daily, weekly or monthly depending on the particular requirements for expression in the subject.

### Treatment of Glaucoma

The present invention provides for the first time higher functioning genetic switches in a retinal model which can be used to regulate transgenes within the eye to treat eye diseases. The SLIM switches described above have the potential to have an increased dynamic range over traditional riboswitches and can be used for the treatment, inhibition or amelioration of one or more symptom of glaucoma, including high intraocular pressure. Prostaglandin synthesis will be regulated by orally ingesting the activating ligand. The dose of the activating ligand will be determined by intraocular pressure (IOP) readings of the patient through the use of a rebound tonometer.

The present disclosure provides methods of treating glaucoma comprising gene therapy using an adeno-associated virus encoding the construct comprising a SLIM switch and one or more genes that regulate prostaglandin synthesis as described herein. In another embodiment, the disclosure provides a method of treating glaucoma comprising administering an adeno-associated virus encoding a construct comprising one or more genes that regulate prostaglandin synthesis and at least one ON-type riboswitch (SLIM). FIGS. 21 and 23 provide one such example of the AAV vector encoding genes necessary for regulation of prostaglandin synthesis and suitable SLIM riboswitches for use in the present invention.

Glaucoma is typified by elevated intra-ocular pressure (IOP) leading to progressive loss of retinal ganglion cells and, ultimately severe visual impairment. Increased IOP results from an imbalance between the production of aqueous humour by the ciliary body in the eye's posterior chamber and its drainage through the trabecular meshwork in the anterior chamber. Glaucoma can be categorized based on whether the drainage through the trabecular meshwork is completely (closed angle) or partially (open angle) blocked. Open angel glaucoma is most common and usually presents with no symptoms other than slow progressive vision loss. Closed angle glaucoma is loss common and is considered to be a medical emergency, presenting with acute eye pain, headaches, blurred vision, excessive lacrimation nausea and vomiting.

Due to its chronic nature, the proposed rAAV.SLIM.PTGS2-P2-PTGFR (SEQ ID NO:81) technology would be appropriate for the treatment of glaucoma.

In one embodiment, a method of reducing, inhibiting or ameliorating at least one symptom of glaucoma is provided. The method comprises administering an exogenous nucleic acid construct to the eye of the subject. In one embodiment, the exogenous nucleic acid construct encodes a transgene that regulates prostaglandin 2α synthesis and at least one smiRNA riboswitch. In another embodiment, the exogenous nucleic acid construct encodes: (i) a transgene that regulates prostaglandin synthesis (e.g., prostaglandin endoperoxide synthase 2 (PTGS2); (ii) a smiRNA switch located within the untranslated region of the transgene, wherein the smiRNA switch comprises at least two riboswitches flanking a pri-miRNA, each riboswitch comprising an aptamer operably linked to an expression platform, and (iii) at least one miRNA target sequence complementary to at least a portion of the pri-miRNA, wherein the transgene is incorporated into cells of the subject and express the transgene, and (b) administering a therapeutically effective amount of the ligand that is able to bind to the aptamer in order to regulate expression of the transgene within the eye to reduce, inhibit or ameliorate at least one symptom of glaucoma. In one embodiment, the symptom is high intraocular pressure.

Suitable ligands include ligands that can cross the blood retinal barrier, as described herein.

Suitably, the transgene that regulates prostaglandin 2α synthesis includes, but is not limited to, for example, PTGS2 (SEQ ID NO:52) among others. Other suitable transgenes are known in the art.

### Kits

This disclosure provides kits. The kits can be suitable for use in the methods described herein. In one aspects, a kit can include a rAAV vector comprising the constructs as described herein, for example, a SLIM switch containing construct. In some aspects, the kit can include a construct comprising rAAV vector encoding a VEGF inhibitor as described herein. In other aspects, the kit can include a construct comprising a rAAV vector encoding one or more genes involved in the regulation of prostaglandin synthesis as described herein. Further, the kits may comprise one or more doses of the ligand to be administered after initial administration of the rAAV vector. Instructions on the timing of the dosages and proper administration methods may be provided.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The term “treating” or “treatment” includes, but is not limited to, reducing, inhibiting or preventing one or more signs or symptoms associated with the disease or disorder. For example, treating glaucoma include, for example, reduction in the intraocular eye pressure (e.g. the symptom of glaucoma being treated is high intraocular pressure).

The terms “effective amount” or “therapeutically effective amount” refer to an amount sufficient to effect beneficial or desirable biological and/or clinical results.

“Expression platform” Within the context of the disclosure, the portion of the modified riboswitch which mediates the effect on the nucleic acid expression is known as the expression platform. Preferably, the expression platform is operably linked to the aptamer domain of the riboswitch, preferably structurally linked, most preferably by a nucleic acid linker. Most preferred is that the aptamer domain is linked to the expression platform by a nucleic acid sequence. Preferably, the stem configuration of the portion of the expression platform changes configuration upon binding of a ligand, such that a change in the configuration of the stem structure results in a corresponding change in the structure of the expression platform, between a first configuration which enhances expression of the nucleic acid sequence and a second configuration which inhibits expression of the nucleic acid sequence.

“Genetic construct” can include nucleic acid sequences that permit it to replicate in the host cell. Examples include, but are not limited to a plasmid, cosmid, bacteriophage, or virus that carries exogenous DNA into a cell. A genetic construct can also include additional selectable marker genes and other genetic elements known in the art. A genetic construct can preferably transduce, transform or infect a cell, thereby causing the cell to express the nucleic acids and/or proteins encoded by the vector.

“Operably linked” a first element is operably linked with a second element when the first element is placed in a functional relationship with functional relationship with the second element. For instance, a aptamer is operably linked to an expression platform when the binding of the aptamer to its ligand causes conformational changes to the expression platform which alters the function of the expression platform (for example, if the expression platform is a ribozyme, binding of the aptamer to the ligand can activate the ribozyme activity). Generally, operably linked DNA sequences are contiguous and, where necessary to join two protein coding regions, in the same reading frame.

It is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### EXAMPLES

### Example 1: Ability to Regulate Gene Expression In Vivo in the Eye

This Example demonstrates that six riboswitches respond to a ligand in cell culture and in vivo when deliver to the mouse retina.

Six small (˜100 bp) riboswitches (K19, Tc40×45, GuaM8HDV, L2Bulge18tc, L2Bulge9 and Theo6HDV) responded to a ligand in cell culture, and in vivo when delivered to the mouse retina using a rAAV2 vector.

The riboswitches were evaluated in HEK293T cells to determine optimal copy number (largest dynamic range) and dose-responsiveness to its activating ligand using a dual luciferase assay. The ligand used were tetracycline and theophylline.

Cell culture experiments revealed significant changes in firefly luminescence in response to dosing of the appropriate ligand (p<0.01, One-way ANOVA, N=4 all groups).

Next, the optimal copy number of each riboswitch was cloned into a rAAV GFP reporter cassette and packaged in an AAV2 capsid. Each GFP-riboswitch cassette was injected intravitreally into C57Bl/6j mice in combination with an AAV2 control vector harboring a non-inducible mCherry reporter gene. Four weeks post-injection, mCherry and GFP fluorescence levels were quantified in vivo using a custom ‘Multiline’ confocal scanning laser ophthalmoscope (cSLO). Mice subsequently received a dose of 1000 mg/kg of its activating ligand (tetracycline, theophylline), and fluorescence levels were quantified 2 and 24 hours post-gavage.

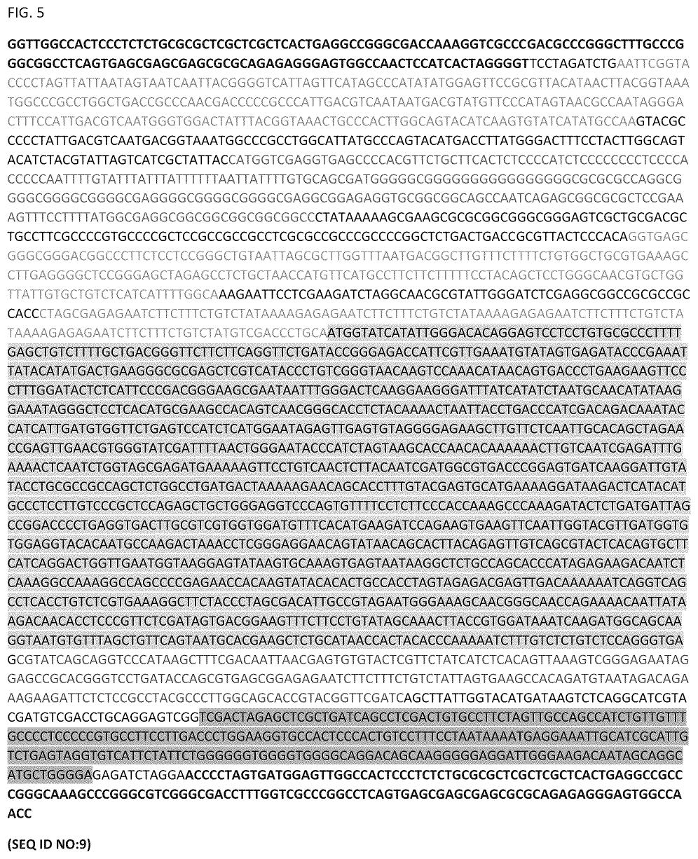

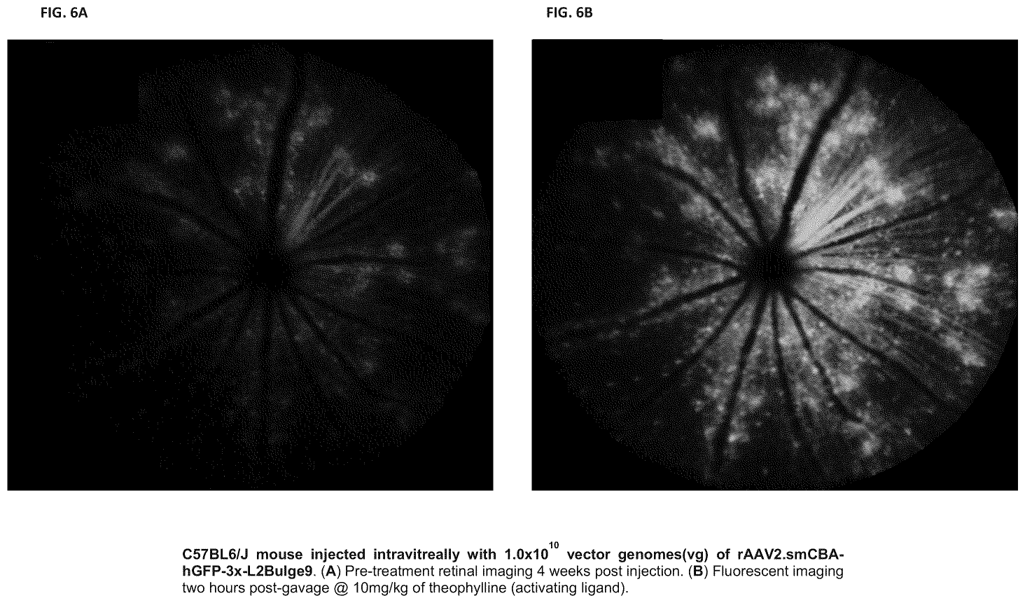

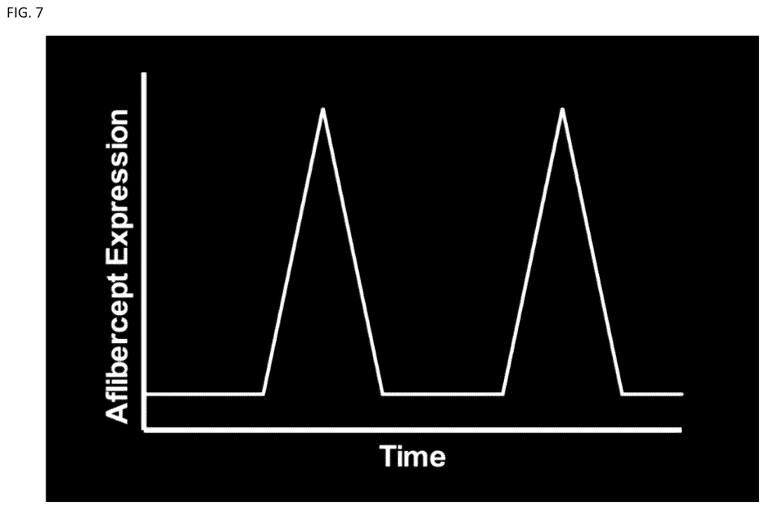

In vivo results demonstrated dosing mice with the activating ligand of each riboswitch could achieve a highly significant change in GFP fluorescence at 2 hours post-gavage compared to pre-treatment levels (p<0.01, paired t test, N=6) (FIG. 6A and 6B). Importantly, GFP fluorescence was recovered to pre-treatment levels 24 hours after receiving the activating ligand. This shows that genes can be delivered and expressed in the retina of the eye.

### Example 2: Evaluation of Optimal Copy Number of Riboswitches and Dynamic Range

### Example 2A: Testing of Known Riboswitches

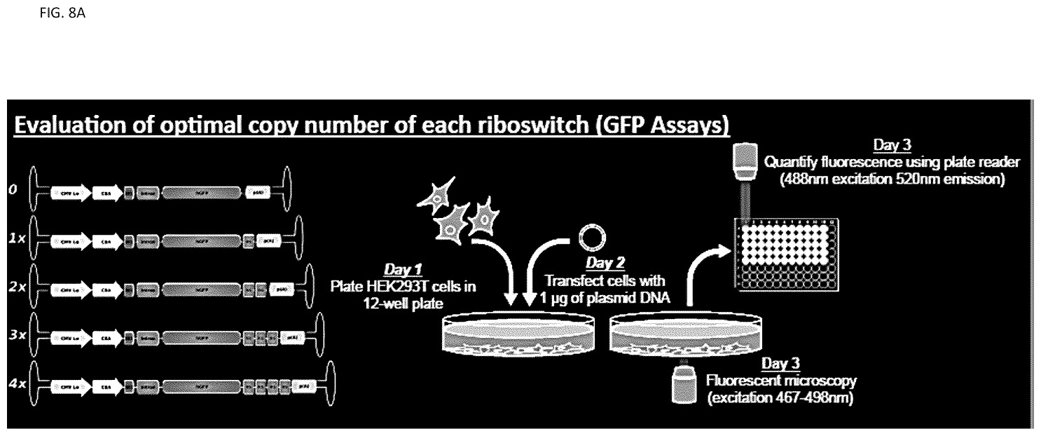

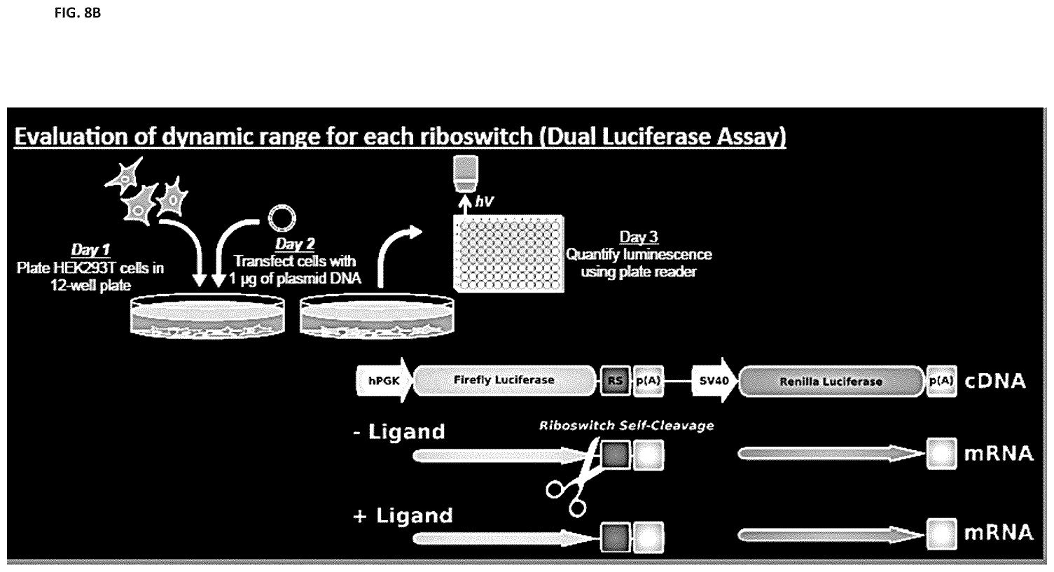

This Example demonstrates the optimal number of riboswitches that can be used in a construct and the related dynamic ranges. The protocol for both assays are outlined in FIG. 8A and 8B. For determining optimal copy numbers, plasmids containing 0-4 copies of each riboswitch were created with the transgene as green fluorescence protein (GFP) for readout. HEK293T cells were plated in a 12-well plate and on day 2 transfected with 1 μg of each plasmid DNA. On day 3, cells were observed by fluorescent microscopy (excitation 467-498 nm) and fluorescence was quantified using a plate reader (488 nm excitation 520 nm emission).

For determining dynamic range, constructs containing firefly luciferase containing the optimal copy number of the riboswitch were made. HEK293T cells were plated on day 1, and transfected on day 2 with 1 μg of plasmid DNA. Cells were treated with 0-100 μM of the ligand corresponding to the riboswitch and the luminescence was quantified on day 3 using plate reader. Results are shown in Table 2.

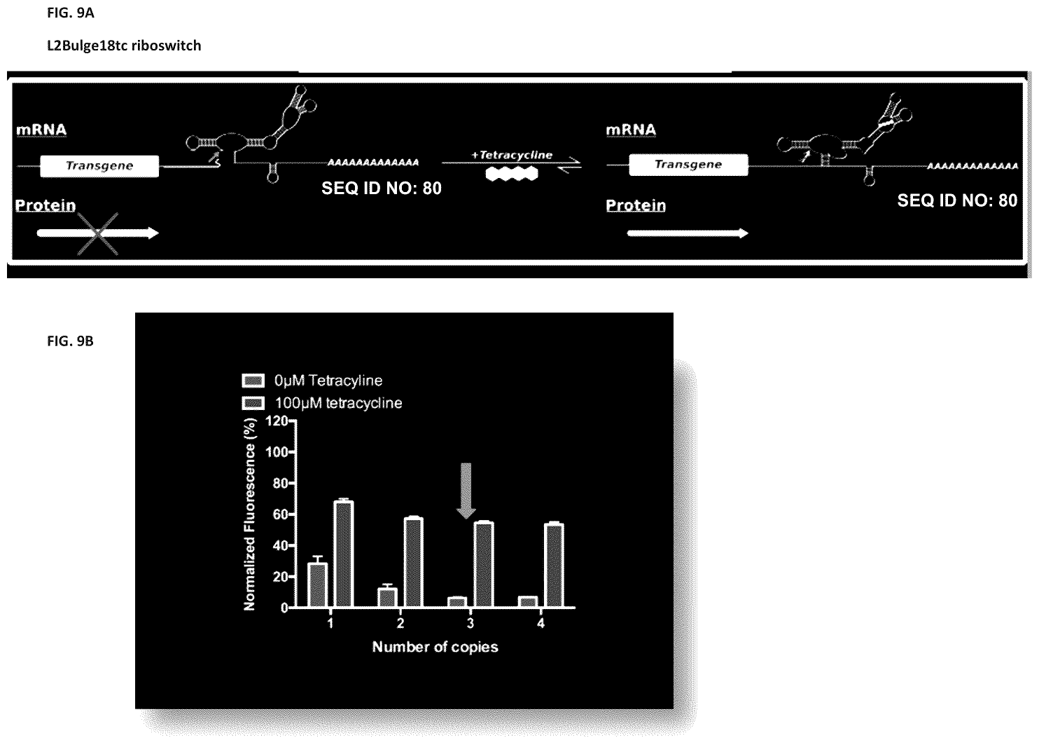

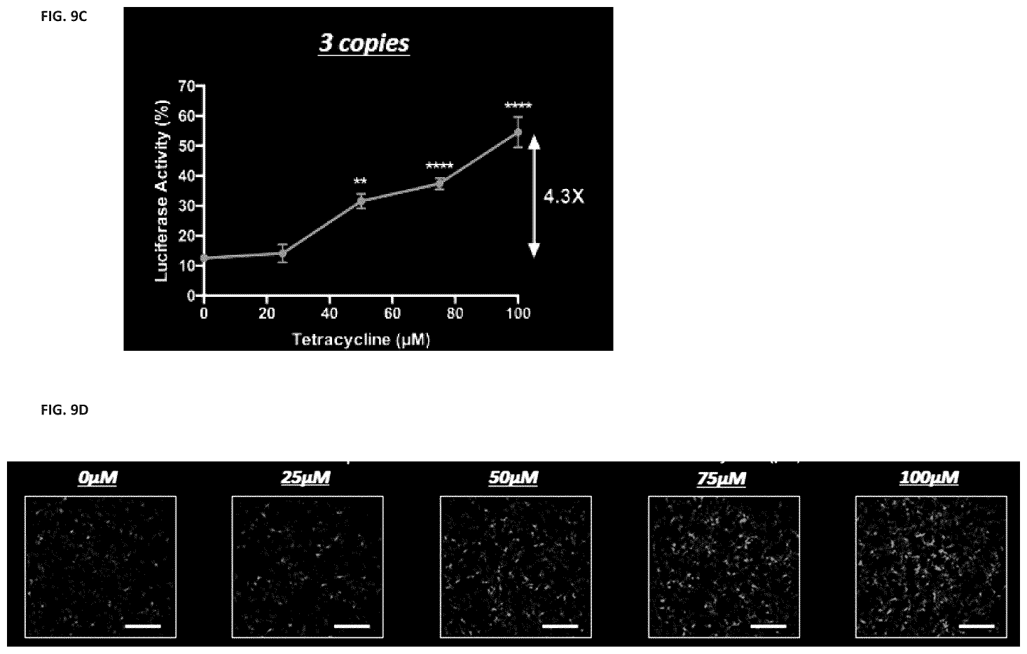

As depicted in FIG. 9A, L2Bulge18tc riboswitch was one ON-type riboswitch tested. The cells were treated with 0 μM, 25 μM, 50 μM, 75 μM, and 100 μM of tetracycline (ligand), with the fluorescent microscopy results shown in FIG. 9D. Fluorescence was quantified and results are demonstrated in FIG. 9B, demonstrating 3 copies provides optimal dynamic range, as depicted in FIG. 9C.

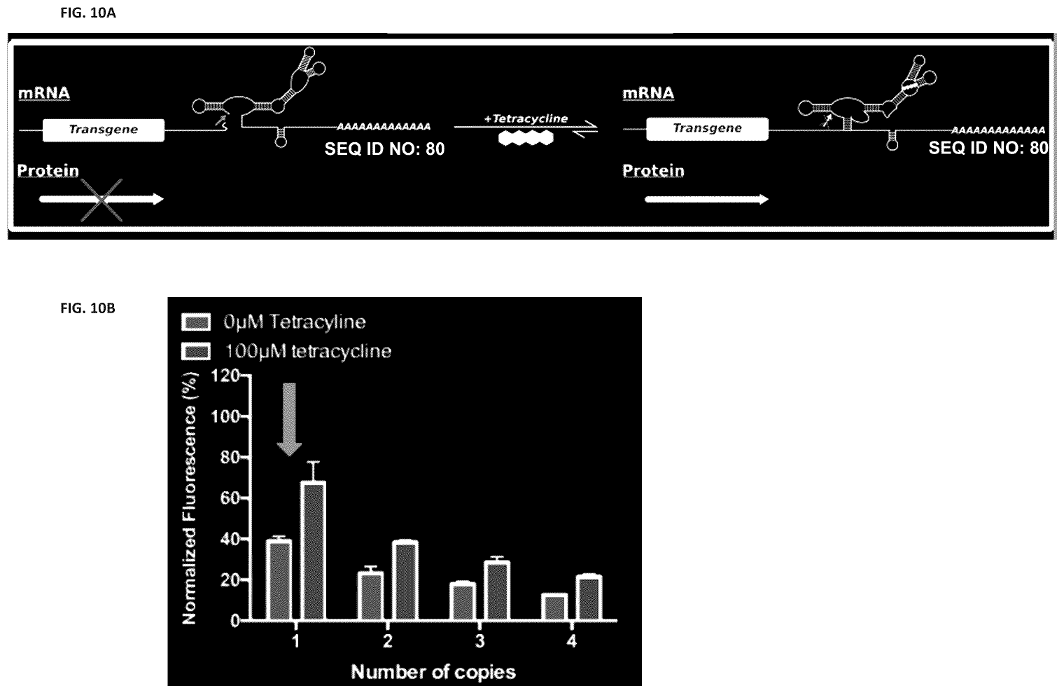

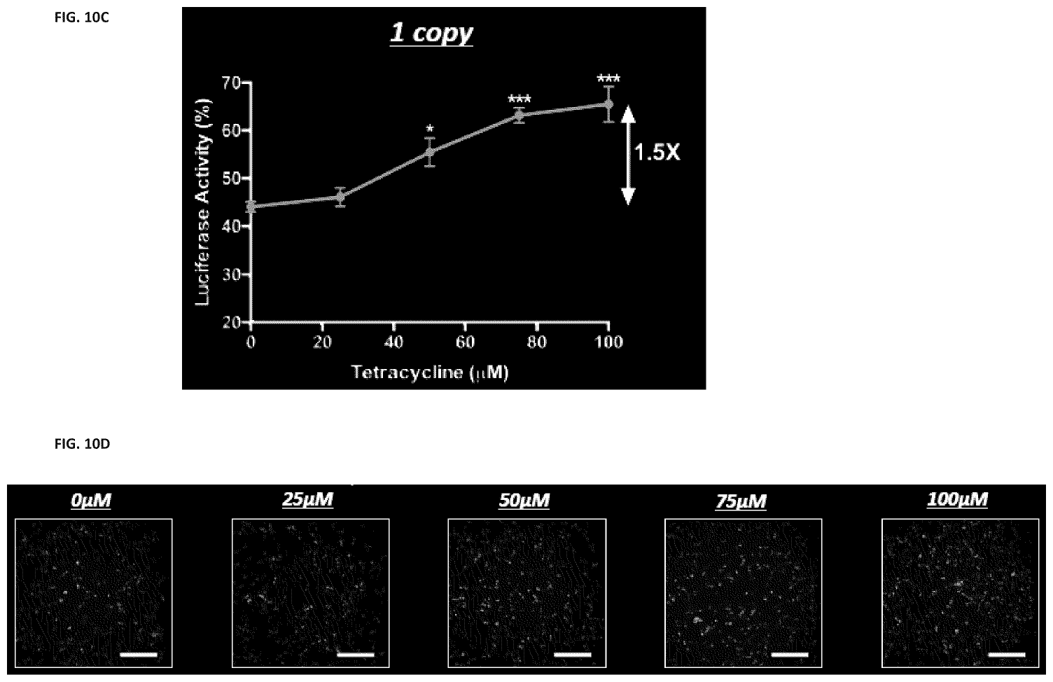

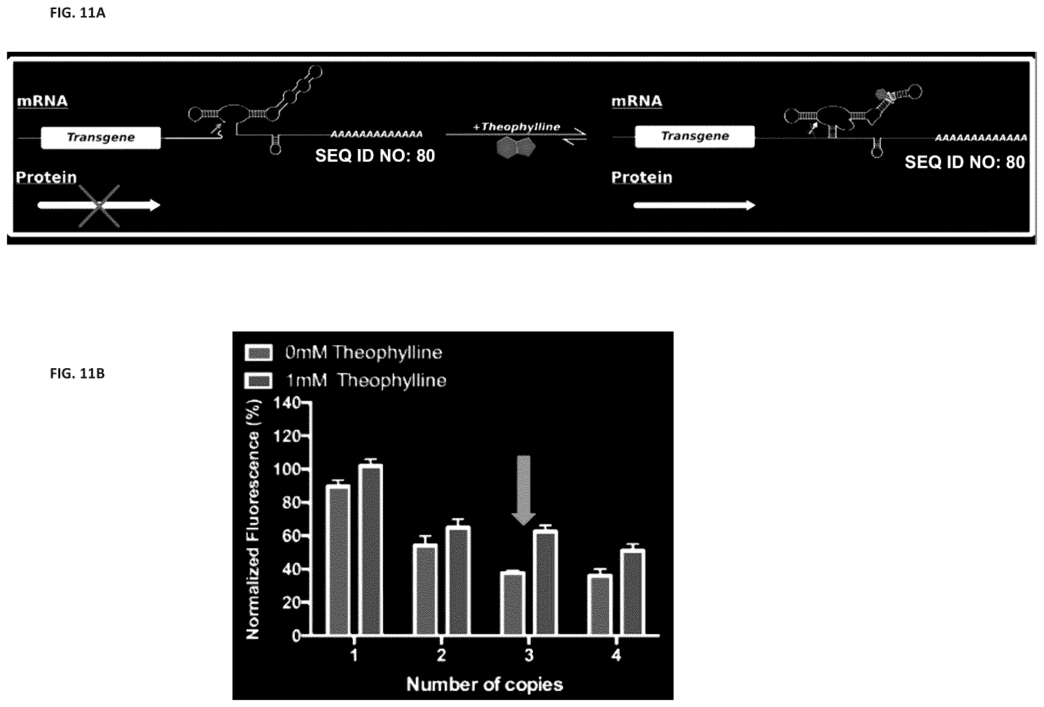

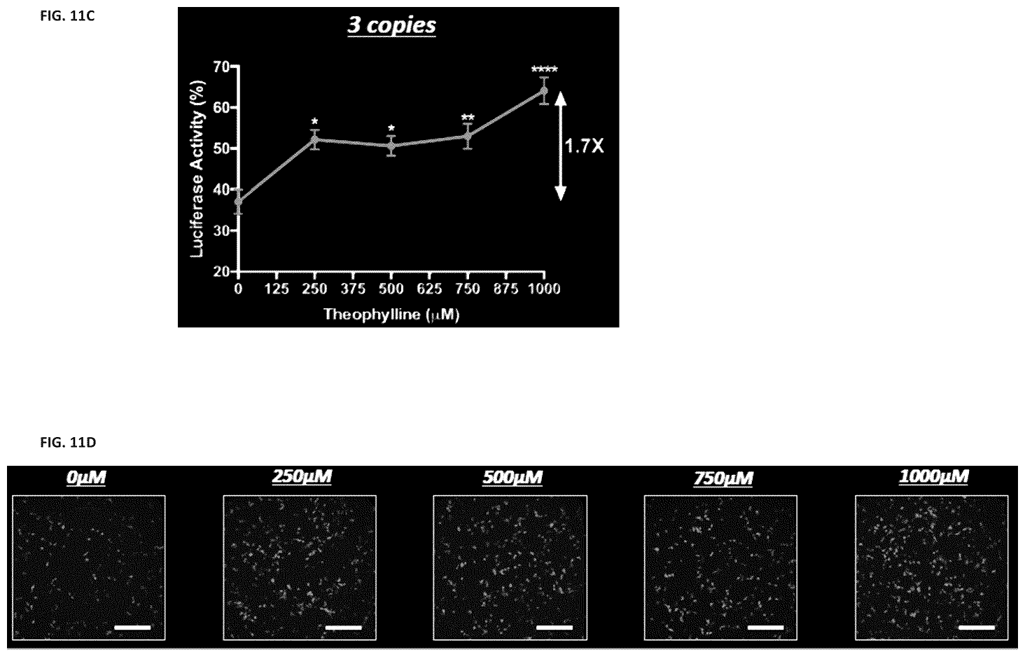

K19 riboswitch was also tested (FIG. 10) as described above, and the results are shown in FIG. 9B-D, demonstrating that 1 copy is the optimal copy number.

L2Bulge 9 was also tested (using activating ligand Theophylline), and the optimal copy number was determined to be 3.

Results for a number of ON-type riboswitches are summarized in Table 1 above.

### Example 2B: Testing of New SLIM Switches

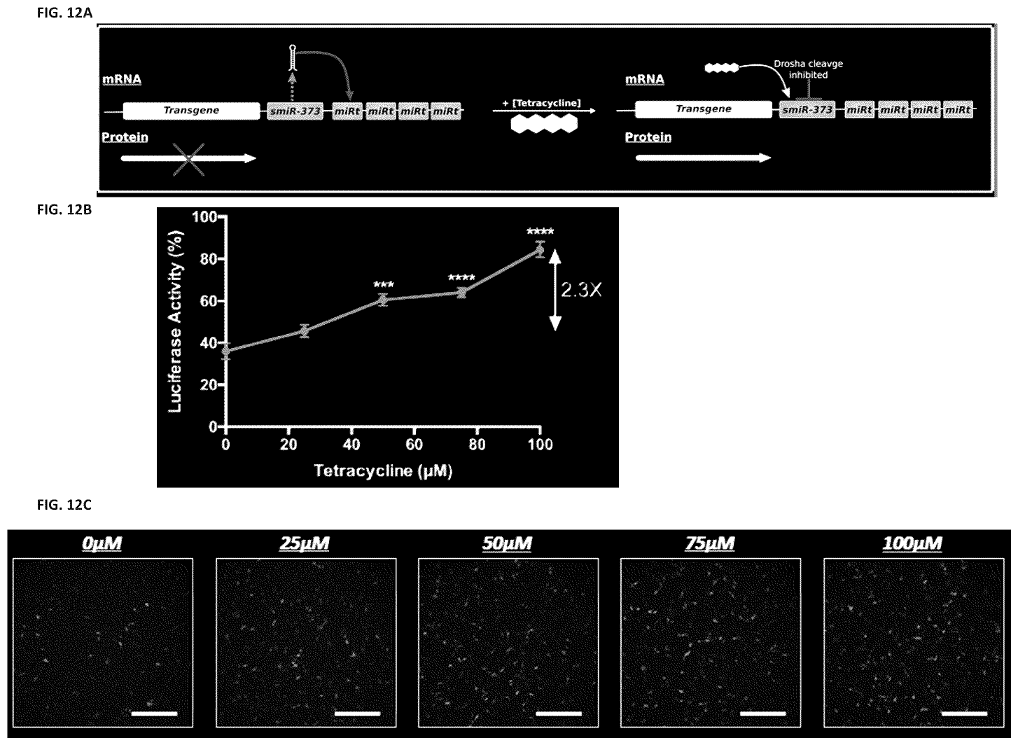

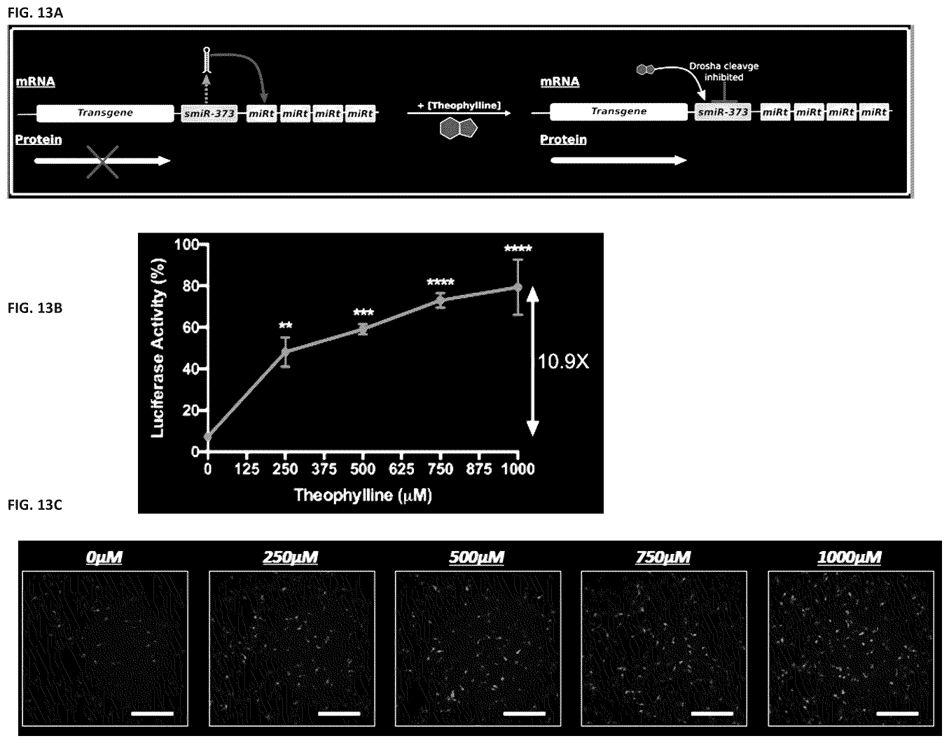

Experiments as described in Example 2A were also carried out using the newly designed Tet-SLIM and Theo-SLIM switches including target miRNA sequence, as depicted in FIG. 12A and 13A, respectively. FIG. 12B and 13B show the dynamic range for both switches, while FIG. 12C and 13C depict fluorescence as seen in the cells treated with 0-100 μM of their respective ligands. Both SLIM riboswitches had an increased dynamic range over similar riboswitches known in the art.

### Example 3: Mouse Study Using SLIM Containing Anti-VEGF Compound

Adult (>2 months old) wild-type (e.g. C57B1/6j strain) mice (n=20) will be purchased from an approved supplier (e.g. Jackson Laboratories) and group housed at the Medical College of Wisconsin in standard conditions. After a period of acclimatization, each animal will undergo bilateral intra-vitreal injections. One eye will receive 2 μl sterile buffer (HBSS+0.014% tween-20) containing purified recombinant adeno-associated virus (rAAV) packaging the cDNA sequences required for biosynthesis of a vascular endothelial growth factor (VEGF) inhibitor (e.g. Aflibercept (Eylea)) under control the SLIM gene-switch (named herein, rAAV.SLIM.αVEGF). The contralateral eye will receive an injection of buffer only to control for the effects of the surgical intervention. Four weeks will be allowed for incorporation of the rAAV.SLIM.αVEGF vector into cells of the retina; based on preliminary data we expect ganglion cells and Müller glia be effectively transduced. All eyes will be imaged by fluorescein angiography (FA) using a confocal scanning laser ophthalmoscope to establish the baseline integrity of the retinal and choroidal blood vessels. Animals will subsequently be assigned randomly to either the treatment (receives activating ligand) or control (no activating ligand) group. Acute choroidal neovascularization (CNV) will be induced in all eyes by making a small hole in Bruch's membrane with a focused infrared laser beam. The extent of CNV formation will be assessed seven and 14 days thereafter by FA.

It is anticipated that animals of the control group (no ligand=low-level αVEGF expression) will demonstrate CNV formation in the rAAV.SLIM.αVEGF injected eyes that is similar in extent to the contralateral buffer injected eyes. By contrast, it is anticipated that animals of the experimental group (with ligand=high-level αVEGF expression) will exhibit significantly reduced CNV formation in rAAV.SLIM.αVEGF injected eyes compared to the contralateral buffer injected eyes. This will serve to demonstrate that rAAV-mediated over-expression of a VEGF inhibitor is an effect method for preventing CNV formation, and that the expression levels of the VEGF inhibitor can be modulated (i.e. increased) through supplementation of the activating ligand, leading to a reduction in CNV formation. All animals will subsequently be euthanized to allow collection of the eyes for biochemical analysis, allowing direct quantification of αVEGF protein levels within treated (with ligand) and control (no ligand) eyes.

### Example 4: Treatment of AMD Using rAAV.SLIM.αVEGF Vector

Due to the slowly progressing nature of AMD, a therapeutic window exists between initial diagnosis and the onset of severe visual impairment in which to intervene. Following diagnosis, patients would receive a single intravitreal injection of the rAAV.SLIM.αVEGF vector suspended in a physiologically relevant buffer. The current treatment paradigm for AMD patients involves monthly or bimonthly intravitreal injections of anti-VEGF protein; a single-dose administration rAAV.SLIM.αVEGF would therefore represent a significantly less invasive treatment alternative. Intravitreal injections of anti-VEGF protein are currently performed under local anesthesia as an outpatient procedure; it is anticipated that this would also be the case for intravitreal administration of the rAAV.SLIM.αVEGF vector. Four to eight weeks would be allowed for incorporation of the rAAV.SLAM.F2α vector into cells of the inner retina. The patient could continue to receive conventional anti-VEGF therapy during this time. The SLIM technology is an ON-type switch; as such anti-VEGF protein will not be expressed in the patient's eye until the activating ligand is provided. Anti-VEGF protein expression levels in the patient eye will be regulated through oral administration of the activating ligand (e.g. in tablet form). The SLIM technology is dose-dependent, allowing the patient/physician to precisely modulate anti-VEGF expression levels within the eye. The retinal cells targeted as part of this procedure do not divide. As a consequence, following incorporation of the vector into those cells, it is anticipated that the rAAV.SLIM.αVEGF vector will persist throughout the patient's lifetime. Anti-VEGF expression can therefore be induced at any time throughout the patient's lifetime by administration of the activating ligand, negating the need for repetitive intra-ocular injections.

### Example 5: AAV-Riboswitch for Treating AMD

Current treatment methodologies for treating AMD focus on the administration soluble receptors or neutralizing antibodies raised against VEGF-A protein in order to inhibit its pro-angiogenic function. Eylea (aflibercept), a recombinant fusion VEGF trap licensed by the Food and Drug Administration for the treatment of wet AMD in 2011, has been found to be highly effective at treating CNV in patients with wet AMD. Although this approach has been largely successful at preventing CNV formation, it requires a monthly high-dose intravitreal injection of anti-VEGF agents throughout a patient's lifetime, resulting in a substantial financial and economic burden. Moreover, continuous bolus administration of both Eylea and Lucentis over multiple years has been shown to accelerate the rate of retinal and choroidal atrophy. Patients are also at an increased risk for injection related complications such as endophthalmitis and cataract formation. In this Example, we combine several technologies developed within our laboratory to create an inducible rAAV-based gene therapy approach to treat wet AMD following a single intravitreal administration.

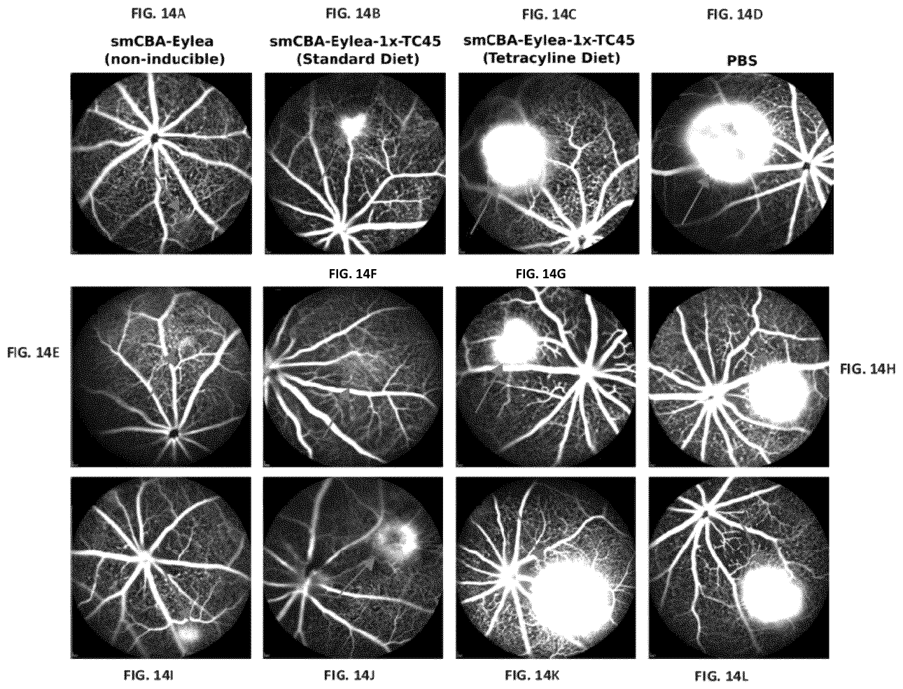

We assessed whether rAAV-mediated over-expression of Eylea is capable of preventing CNV formation following laser injury to Bruch's membrane. By incorporating a tetracycline-responsive riboswitch (1xTC45) riboswitch in the 3′-UTR of the expression cassette, we were also able to address whether modulating the intraocular concentration of Eylea led to an alteration in the severity of CNV lesions observed. To this end, age-matched C57BL/6J mice were unilaterally injected with cither PBS (n=10), 1.0×10¹⁰vg of rAAV2/2[MAX].smCBA-Eylea (n=10) or 1.0×10¹⁰vg rAAV2/2[MAX].smCBA-Eylea-1x-TC45 (n=20). Immediately following injections, half of the mice (n=10) injected with rAAV2/2[MAX].smCBA-Eylea-1×-TC45 were placed on diet containing 50 mg/g tetracycline. Six weeks post-injection, CNV formation was initiated by rupturing Bruch's membrane using an infrared laser diode (see methods). Seven days following laser injury, neovascular lesion size and leakage was assessed via fluorescein angiography using cSLO imaging. The majority of mice ubiquitously expressing Eylea (smCBA-Eylea) either did not develop lesions at the site of laser injury, or developed small grade 1 or 2A type lesions that leaked minimal fluorescein, even after a 5-minute period. (FIG. 14A-C). Mice injected with the ‘OFF-type’ smCBA-Eylea-1x-TC45 vector and placed on regular diet also predominantly developed only minor lesions, though a small increase in the number of 2B lesions were observed compared to the non-inducible Eylea construct (FIG. 14D-F). Lowering Eylea expression through activation of the TC45 riboswitch greatly increased the severity of CNV lesions (FIG. 14G-I) to the extent that they were similar in extent to PBS-sham injected mice (FIG. 14L-J).

Lesion images were graded by three blinded scientists using the grading system described by Krzystolik et al. (see methods for details). Importantly, grade distribution was significantly different for each treatment group Mice receiving a sham injection had the highest incidence of clinically significant ‘Grade 2B’ lesions, while mice ubiquitously over-expressing Eylca (smCBA-Eylea) had the lowest incidence of ‘Grade 2B’ lesions.

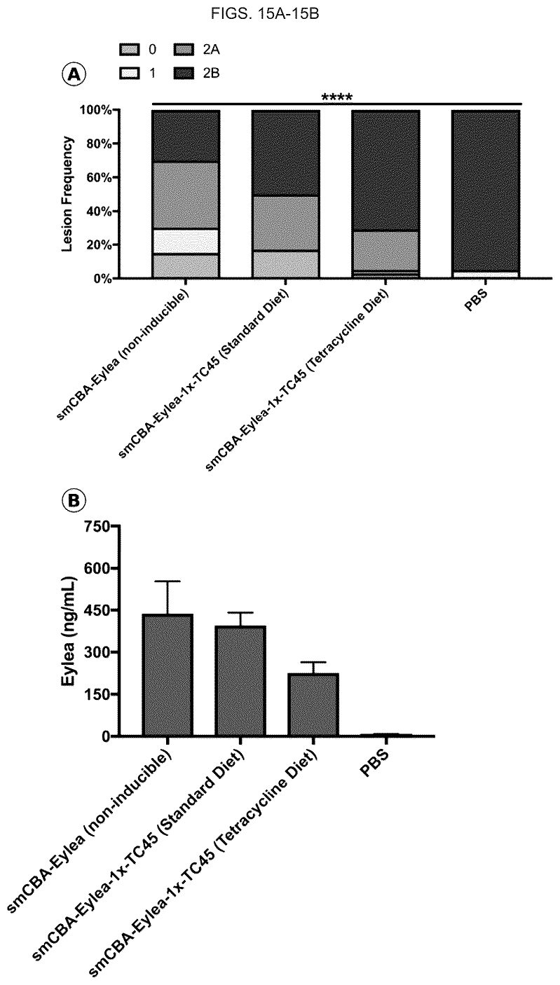

Notably, downregulating Eylea expression through activation of the TC45 riboswitch resulted in a considerable increase in the incidence of ‘Grade 2B’ lesions compared to rAAV2[MAX].smCBA-Eylea-1x-TC45 injected mice receiving standard diet (FIG. 15A). Finally, we determined the levels of non-complexed Eylea in each sample using an Eylea-specific ELISA (Eagle Biosciences). As expected, eyes injected with the non-inducible construct (smCBA-Eylea) contained the highest levels of free Eylea (438 ng/ml). Moreover, high levels of free Eylea were detected in animals injected with vector containing the tunable construct (395 ng/mL), though levels were lower than animals injected with the non-inducible construct. Importantly, tetracycline-mediated activation of the TC45 riboswitch resulted in a significant 1.75-fold decrease in non-complexed Eylea (p<0.05). (FIG. 15B) The levels of free Eylea correlated strongly with the occurrence of clinically significant lesions.

### Example 6: Self-Targeting Ligand Inactivating miRNAs for AMD Treatment in Humans

A SLIM switch requires the basal region of a pri-miRNA to be replaced with an aptamer. This sequence is cloned into either the 3′-or 5′ untranslated region of an expression cassette. Additionally, miRNA target sites that are complementary to the sequence of the mature miRNA can be included in one or multiple copies at either the 5′ or 3′ untranslated region of the cassette.

Self-targeting Ligand Inactivated miRNAs (SLIM) switches function by regulating gene expression at the post-transcriptional level. In conditions when the activating ligand is absent, the pri-miRNA will be cleaved by drosha from the nascent transcript. This miRNA will be processed and act as the second mechanism of gene silencing, through binding of the complementary target sites. When the activating ligand is provided, gene expression is unaltered.

Multiple copies of each riboswitch (SLIM) can be included into the 3′-untranslated region of the gene of interest. Each riboswitch appears to have an optimal number of copies as shown in Table 2. Furthermore, multiple copies of miRNA target sites can be included in the 3′ or 5′ untranslated region.

Due to the slowly progressing nature of AMD, a therapeutic window exists between initial diagnosis and the onset of severe visual impairment in which to intervene. Following diagnosis, patients would receive a single intravitreal injection of the rAAV.SLIM.αVEGF vector suspended in a physiologically relevant buffer. The current treatment paradigm for AMD patients involves monthly or bimonthly intravitreal injections of anti-VEGF protein. Thus, a single-dose administration rAAV.SLIM.αVEGF would therefore represent a significantly less invasive treatment alternative. Intravitreal injections of anti-VEGF protein are currently performed under local anaesthesia as an outpatient procedure; it is anticipated that this would also be the case for intravitreal administration of the rAAV.SLIM.αVEGF vector. The patient could continue to receive conventional anti-VEGF therapy during this time. The SLIM technology is an ON-type switch; as such anti-VEGF protein will not be expressed in the patient's eye until the activating ligand is provided. Anti-VEGF protein expression levels in the patient eye will be regulated through oral administration of the activating ligand (e.g. in tablet form). The SLIM technology is dose-dependent, allowing the patient/physician to precisely modulate anti-VEGF expression levels within the eye. The retinal cells targeted as part of this procedure do not divide. As a consequence, following incorporation of the vector into those cells, it is anticipated that the rAAV.SLIM.αVEGF vector will persist throughout the patient's lifetime. Anti-VEGF expression can therefore be induced at any time throughout the patient's lifetime by administration of the activating ligand, negating the need for repetitive intra-ocular injections.

### Treating Glaucoma with Vectors Comprising Riboswitches

Primary open angle glaucoma (POAG) is the second leading cause of irreversible blindness worldwide and is characterized by progressive loss of retinal ganglion cells and atrophy of the optic nerve, leading to visual field deficits. The major risk factor for POAG is increased intraocular pressure (IOP) resulting from decreased aqueous humor outflow. The gold standard clinical therapy for glaucoma is to reduce IOP through a combination of topical drug administration, and in late-stage disease, surgery. Unfortunately, due to the largely asymptomatic (i.e. non-painful) nature of glaucoma and the necessity to maintain a lifelong daily treatment regimen, patient compliance with drug therapies is extremely poor (<20%), leading to the development of severe sight-threatening complications, even in patients diagnosed early.

### AAV-Riboswitch Experiments—a Model For Treating Glaucoma

Prostaglandin analogs (e.g. Latanprost) are administered clinically as esterified pro-drugs that are absorbed across the corneal epithelium and are hydrolyzed into their active form as they pass through the stroma and corneal endothelium before diffusing into the aqueous humor. Binding of soluble PGF_{2α} analog to PTGFR triggers up-regulation of matrix metaloprotinase (MMP) production within the ciliary muscle that promotes remodelling of the extracellular matrix and increased uveoseleral aqueous outflow. Native PGF_{2α} is enzymatically derived from aracadonic acid, which is present in the cornea at high levels, via a multistep process.

De novo biosynthesis and secretion of PGF_{2α} from the cornea into the anterior chamber promises to be an effective strategy for lowering IOP that improves upon the current gold-standard clinical treatment approach. We have demonstrated that over-expression of prostaglandin endoperoxide synthase 2 (PTGS2), the rate limiting enzyme in PGF_{2α}0 biosynthesis, and PTGFR, causes a significant decrease in IOP over a period of three months. The cDNA sequences (below) for both enzymes were incorporated into a AAV expression construct (complete sequence below) incorporating either no riboswitch (non-inducible) or with a 3′ TC40 and 5′ TC45 tetracycline inducible riboswitch Mice received intracamereal injections of PBS (N=10), rAAV.smCBA-PTGFR.P2A.PTGFR (N=10, non-inducible) or rAAV.smCBA-TC40-PTGFR.P2A.PTGFR-TC45 (inducible). IOP was measured by rebound tonometry before and 12 weeks following injection. Half of the inducible mice were placed on tetracycline containing diet for that period. Mice placed on diet (PGF2alpha Expression OFF) showed no decreased in intraocular pressure over the 12 week period. Mice on normal diet (PGF2alpha expression ON) showed a highly significant decrease in IOP, similar to when PGF2alpha is constitutively expressed (no switch).

### Sequence Listing Statement and Reference to a Sequence Listing Submitted Via Patent Center

The application includes the sequence listing through-out the specification and in the attached sequence listing. The content of the ST26 format sequence listing named “650053_00984.xml” which is 136,298 bytes in size and was created on Jul. 21, 2023 and electronically submitted via Patent Center herewith the application is incorporated herein by reference in its entirety. A few sequences are listed below, but are not to be considered a full listing.

L2bulge9 (Theophylline OFF switch)
(SEQ ID NO: 11)
CTCGAGGCGATCGCAAACAAACAAAGCTGTCACCGGATGTGCTTTCCG
GTCTGATGAGTCCGTTGTCCAATACCAGCATCGTCTTGATGCCCTTGG
CAGTGGATGGGGACGGAGGACGAAACAGCAAAAAGAAAAATAAAAATT
TTTTTTTTAATTAATCTTGGGCCC
L2bulgel8tc (Tetracycline ON switch)
(SEQ ID NO: 12)
CTCGAGGCGATCGCAAACAAACAAAGCTGTCACCGGATGTGCTTTCCG
GTCTGATGAGTCCGTTGTCCAAAACATACCAGATTTCGATCTGGAGAG
GTGAAGAATTCGACCACCTGGACGAGGACGGAGGACGAAACAGCAAAA
AGAAAAATAAAAATTAATTAATCTTGGGCCC
Theo6HDV (Theophylline OFF switch)
(SEQ ID NO: 13)
ATGGCCGGCATGGTCCCAGCCTCCTCGCTGGCGCCGGCTGGGCAACCA
CATACCAGCCGAAAGGCCCTTGGCAGGTGGGCGAATGGGACGCACAAA
TCTCTCTAGCTTCCCAGAGAGAAGCGAGAGAAAAGTGGCTCTC
GuaM8HDV (Guanine OFF switch)
(SEQ ID NO: 14)
ATGGCCGGCATGGTCCCAGCCTCCTCGCTGGCGCCGGCTGGGCAATGC
TATAATCGCGTGGATATGGCACGCAAGTTTCTACCGGGCACCGTAAAT
GTCCGACTAGTAGCGAATGGGACGCACAAATCTCTCTAG
K19 (Tetracycline ON switch)
(SEQ ID NO: 15)
CAAACAAACAAAGGCGCGTCCTGGATTCGTGGTAAAACATACCAGATT
TCGATCTGGAGAGGTGAAGAATACGACCACCTGTAGTATCCAGCTGAT
GAGTCCCAAATAGGACGAAACGCGCTAAACAAACAAAC
TC40 (Tetracycline OFF switch)
(SEQ ID NO: 16)
CTGAGGTGCAGGTACATCCAGCTGACGAGTCCCAAATAGGACGAAAGG
GAGAGGTGAAGAATACGACCACCTAGGCTCGAAAGAGCCTAAAACATA
CCTTTCCTGGATTCCACTGCTATCCAC
Tet-SLIM
(SEQ ID NO: 17)
CTAGCACGGGTCCCTAAAACATACCGTGAGCGCGAAAGCGCCCCCATT
TTGAGTTAGTGAAGCCACAGATGTAACTCAAAATGGGGGCGCTTTCCC
GCCTACGGAGAGGTGAAGAATACGACCACCTAGAAGCTTATTGGTACA
TGATAACACCCCAAAATCGAAGCACTTCAAAAACACCCCAAAATCGAA
GCACTTCAAAAACACCCCAAAATCGAAGCACTTCAAAAACACCCCAAA
ATCGAAGCACTTCAGTCTCAGGCATCGTACGATGTCGACCTGCAGG

We claim:

1-52. (canceled)

53. A nucleic acid construct comprising SEQ ID NO: 52, 53, or 54.

54. The nucleic acid construct of claim 53, wherein the nucleic acid construct comprises SEQ ID NO: 52.

55. The nucleic acid construct of claim 54, further comprising a nucleic acid encoding a porcine teschovirus-1 2A (P2A) sequence.

56. The nucleic acid construct of claim 55, wherein the nucleic acid encoding a porcine teschovirus-1 2A (P2A) sequence is SEQ ID NO: 61.

57. The nucleic acid construct of claim 53, wherein the nucleic acid construct comprises SEQ ID NO: 53.

58. The nucleic acid construct of claim 57, further comprising a nucleic acid encoding a porcine teschovirus-1 2A (P2A) sequence.

59. The nucleic acid construct of claim 58, wherein the nucleic acid encoding a porcine teschovirus-1 2A (P2A) sequence is SEQ ID NO: 61.

60. The nucleic acid construct of claim 53, wherein the nucleic acid construct comprises SEQ ID NOs: 52 and 53.

61. The nucleic acid construct of claim 60, further comprising a nucleic acid encoding a porcine teschovirus-1 2A (P2A) sequence.

62. The nucleic acid construct of claim 61, wherein the nucleic acid encoding a porcine teschovirus-1 2A (P2A) sequence is SEQ ID NO: 61.

63. The nucleic acid construct of claim 53, wherein the nucleic acid construct comprises SEQ ID NO: 54.

64. The nucleic acid construct of claim 53, further comprising a promoter or enhancer.

65. The nucleic acid construct of claim 63, further comprising at least one of SEQ ID NOs: 59, 63, and 64-67.

66. The nucleic acid construct of claim 53, further comprising at least one of SEQ ID NOs: 59, 63, and 64-67.

67. The nucleic acid construct of claim 53, wherein the nucleic acid construct comprises SEQ ID NO: 58.

68. A viral vector comprising the nucleic acid construct of claim 53.

69. The viral vector of claim 68, wherein the viral vector is an adeno-associated virus (rAAV), a lentivirus, an adenovirus, a plasmid, a herpes simplex virus, a baculovirus, or a bacteriophage.

70. The viral vector of claim 69, wherein the viral vector is an adeno-associated virus (AAV).

71. The viral vector of claim 70, wherein the viral vector is an adeno-associated virus serotype 2 (AAV2).
